(19) United States
(12) Patent Application Publication
(10) Pub. No.: US 2022/0195513 A1
(43) Pub. Date: Jun. 23, 2022

(54) SEQUENCING METHOD

(71) Applicant: Sumitomo Chemical Co, Ltd., Tokyo (JP)

(72) Inventors: Jonathan Behrendt, Godmanchester (GB); Kiran Kamtekar, Godmanchester (GB); Martin Humphries, Godmanchester (GB)

(73) Assignee: Sumitomo Chemical Co, Ltd., Tokyo (JP)

(21) Appl. No.: 17/555,151

(22) Filed: Dec. 17, 2021

(30) Foreign Application Priority Data
Dec. 17, 2020 (GB) GB2019966.7

(51) Int. Cl. C12Q 1/6869; C12Q 1/6809

(52) U.S. Cl. CPC C12Q 1/6869; C12Q 1/6809

(57) ABSTRACT

A method for determining the sequence of a polynucleotide is described. The method comprises detecting in a sequencing reaction the incorporation of a first, second, third, or fourth nucleotide using first and second excitation wavelengths and a photodetector having a detection window comprising a range of wavelengths.

### RELATED APPLICATIONS

This application claims foreign priority benefits under 35 U.S.C. § 119(a)-(d) or 35 U.S.C. § 36(b) of British application number GB 2019966.7, filed Dec. 17, 2020, the entirety of which is incorporated herein.

### BACKGROUND

The present disclosure relates to methods for sequencing nucleic acids and reagents for use in such methods.

One of the technologies that have improved the study of nucleic acids is the development of fabricated arrays of immobilised nucleic acids. These arrays consist typically of a high-density matrix of polynucleotides immobilised onto a solid support material. Using such arrays, current sequencing methods allow for the parallel processing of millions or even billions of cloned nucleic acids or nucleic acid fragments in a single sequencing run. These high-throughput approaches to nucleic acid analysis are often referred to as massive parallel sequencing, or next generation sequencing (NGS) methods. NGS technologies differ in precise methodology and sequencing chemistry but share the feature of the parallel analysis of clonally amplified nucleic acid template clusters that are spatially separated and immobilised within a flow cell.

One way of determining the nucleotide sequence of a nucleic acid bound to an array is called “sequencing by synthesis” or “SBS”. This technique requires the incorporation of the correct nucleotide complementary to that of the nucleic acid being sequenced. Thus, each nucleotide residue is identified as it is incorporated into the growing nucleic acid strand. The incorporated nucleotide is read using an appropriate label attached thereto before removal of the label moiety and the subsequent next round of sequencing. Detection of the label can be carried out using various methods, including luminescence spectroscopy or by other optical means. Generally, the preferred label is a fluorophore, which, after absorption of energy, emits radiation at a defined wavelength. SBS methods using 1, 2, and 4 channels have been described, for example, in WO 2015/084985. Nevertheless, luminescence-based sequencing instrumentation is typically large and expensive, and improvements in increased throughput capacity and reduced cost are required.

### SUMMARY

A summary of aspects of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects and/or a combination of aspects that may not be set forth.

The present inventors have found that detecting nucleotides in a sequencing cycle using a single detection filter provides improvements in nucleic acid sequencing speed and throughput capacity relative to current systems, including systems comprising the use of one or two detection channels.

Accordingly, in some embodiments there is provided a method for determining the sequence of a polynucleotide comprising detecting in a sequencing reaction the incorporation of a first, second, third, or fourth nucleotide using radiation of first and second excitation wavelengths and a single photodetector having a detection window comprising a range of wavelengths. At least the first nucleotide, but not the second nucleotide, comprises a first emitter. At least the second nucleotide, but not the first nucleotide, comprises a second emitter. When irradiated with radiation of the first excitation wavelength, the peak intensity of emission of the second emitter in the detection window is ≤20%, preferably ≤10%, of the peak intensity of emission of the first emitter. When irradiated with radiation of the second excitation wavelength, the peak intensity of emission of the first emitter in the detection window is ≤20%, preferably ≤10%, of the peak intensity of emission of the second emitter.

Optionally, when irradiated with radiation of the first excitation wavelength, the peak intensity of emission of the second emitter in the detection window is ≤5%, preferably ≤2%, of the peak intensity of emission of the first emitter. Optionally when irradiated with radiation of the second excitation wavelength, the peak intensity of emission of the first emitter in the detection window is ≤5%, preferably ≤2%, of the peak intensity of emission of the second emitter.

Optionally, the method further comprises first and second imaging events.

Optionally, the detection window of the photodetector is the same in both the first and second imaging events.

Optionally, the first imaging event comprises the provision of radiation of the first excitation wavelength and the second imaging event comprises the provision of radiation of the second excitation wavelength.

Optionally, the first imaging event provides a first luminescent detection pattern at the photodetector and the second imaging event provides a second luminescent detection pattern at the photodetector. The combination of first and second luminescent detection patterns is used identify the nucleotide that is incorporated into the polynucleotide.

The brightness of the emitters used in the disclosed methods may be calculated as the extinction coefficient multiplied by the photoluminescence quantum yield at a specific wavelength. Optionally, at a specific detection wavelength within the detection window, when irradiated with radiation of the first excitation wavelength, the brightness of the first emitter is at least 10× greater than the brightness of the second emitter. In some embodiments, when irradiated with radiation of the first excitation wavelength, the brightness at the specific detection wavelength of the first emitter is at least 20×, 50×, or 100× greater than the brightness of the second emitter.

Optionally, at a specific wavelength within the detection window, when irradiated with radiation of the second excitation wavelength, the brightness of the second emitter is at least 10× greater than the brightness of the first emitter. In some embodiments, when irradiated with radiation of the second excitation wavelength, the brightness at the specific detection wavelength of the second emitter is at least 20×, 50×, or 100× greater than the brightness of the first emitter.

Optionally, the first and/or second excitation wavelength may be 320 nm, 355 nm, 405 nm, 449 nm, 488 nm, 520 nm, 530 nm, 532 nm, 561 nm, 633 nm 635 nm, 640 nm, 650 nm, 660 nm, or 780 nm (in each case, each of these values may be varied by +/−2 nm, 5 nm, or 10 nm). Thus, a plurality of these excitation wavelengths may be used in combination as the first and second excitation wavelengths.

Optionally, the first and second excitation wavelengths differ by at least 50 nm. In some embodiments, the first and second excitation wavelengths differ by at least 70 nm, 100 nm, or 120 nm.

Optionally, the photodetector may comprise a long pass filter or a band pass filter. Preferably, in embodiments comprising a long pass filter, the wavelength detection window may be arranged to detect all wavelengths less than a specified upper limit, which may be, for example 570 nm.

Preferably, in embodiments comprising a band pass filter, the wavelength detection window has a width of ≤60 nm, such as ≤50 nm or ≤40 nm. In some embodiments, the wavelength detection window has a width of ≤30 nm.

Optionally, at least one of the first and second emitters has an emission peak which at 50% peak intensity overlaps by at least 5 nm with the detection window, such as by at least, 10 nm, 15 nm, or 20 nm.

Optionally, at least one of the first and second emitters has an emission peak which at 50% peak intensity encompasses the width of the detection window.

Optionally, at least one of the first and second emitters has an emission spectrum which overlaps with the detection window.

Optionally, at least one of the first and second emitters has an emission spectrum and at least 20% of the integrated intensity of the emission spectrum is within the detection window. For example, in some embodiments, at least 30%, 40%, 50%, 60%, 70%, 80%, or 90% of the integrated intensity of the emission spectrum is within the detection window.

The FWHM (or “full width at half maximum”) may be used to define the emission peak for a given emitter and may be determined as the width of a spectrum curve measured between those points on the y-axis which are half the maximum amplitude. Optionally, at least one of the first and second emitters has an emission peak, measured in terms of the FWHM, which is entirely within the detection window.

Optionally, the difference in absorption peaks of the first emitter and the second emitter is at least 50 nm, such as at least 70 nm, 100 nm, or 120 nm.

Optionally, when excited (i.e. when the relevant excitation wavelength is applied):
the first nucleotide comprises the first emitter but not the second emitter;
the second nucleotide comprises the second emitter but not the first emitter;
the third nucleotide comprises the first and second emitters; and
the fourth nucleotide comprises neither the first nor second emitter.

Optionally, the method further comprises:
a first imaging event after irradiation with radiation of the first excitation wavelength providing a first luminescent detection pattern at the photodetector; and
a second imaging event after irradiation with radiation of the second excitation wavelength providing a second luminescent detection pattern at the photodetector.
   The combination of first and second luminescent detection patterns is used identify the nucleotide that is incorporated into the polynucleotide.

Optionally, a positive signal in the first luminescence detection pattern is indicative of the incorporation of the first or third nucleotide into the polynucleotide; and a positive signal in the second luminescence detection pattern is indicative of the incorporation of the second or third nucleotide into the polynucleotide.

Optionally:
a positive signal in the first but not the second luminescence detection pattern is indicative of the incorporation of the first nucleotide into the polynucleotide;
a positive signal in the second but not the first luminescence detection pattern is indicative of the incorporation of the second nucleotide into the polynucleotide;
a positive signal in both the first and second luminescence detection patterns is indicative of the incorporation of the third nucleotide into the polynucleotide; and
a positive signal in neither the first nor second luminescence detection pattern is indicative of the incorporation of the fourth nucleotide into the polynucleotide.

In some embodiments, the method further comprises the use of a third emitter, and in some of these embodiments, the method comprises the use of third and fourth emitters.

Optionally, in these embodiments, the third and fourth emitters have excitation properties such that:
when irradiated with radiation of the first excitation wavelength, the peak intensity of emission of the second, third, and/or fourth emitters in the detection window is ≤20%, preferably ≤10%, of the peak intensity of emission of the first emitter;
when irradiated with radiation of the second excitation wavelength, the peak intensity of emission of the first, third, and/or fourth emitters in the detection window is ≤20%, preferably ≤10%, of the peak intensity of emission of the second emitter;
when irradiated with radiation of a third excitation wavelength, the peak intensity of emission of the first, second, and/or fourth emitters in the detection window is ≤20%, preferably ≤10%, of the peak intensity of emission of the third emitter in the detection window; and
when irradiated with radiation of a fourth excitation wavelength, the peak intensity of emission of the first, second, and/or third emitters in the detection window is ≤20%, preferably ≤10%, of the peak intensity of emission of the fourth emitter in the detection window.

Optionally, in these embodiments, when excited:
the first nucleotide comprises only the first emitter, and not any of the second, third, or fourth emitters;
the second nucleotide comprises only the second emitter, and not any of the first, third, or fourth emitters;
the third nucleotide comprises only the third emitter, and not any of the first, second, or fourth emitters.
the fourth nucleotide may comprise none of the first, second, third, or fourth emitters, or alternatively, may comprise at least two of the first, second, and third emitters and not the fourth emitter.

The method further comprises:
a first imaging event after irradiation with radiation of the first excitation wavelength providing a first luminescent detection pattern at the photodetector;
a second imaging event after irradiation with radiation of the second excitation wavelength providing a second luminescent detection pattern at the photodetector; and
a third imaging event after irradiation with radiation of the third excitation wavelength providing a third luminescent detection pattern at the photodetector.
   The combination of first, second, and third luminescent detection patterns is used identify the nucleotide that is incorporated into the polynucleotide.

Optionally, in these embodiments, when excited:
the first nucleotide comprises only the first emitter, and not any of the second, third, or fourth emitters;
the second nucleotide comprises only the second emitter, and not any of the first, third, or fourth emitters;
the third nucleotide comprises only the third emitter, and not any of the first, second, or fourth emitters; and
the fourth nucleotide comprises only the fourth emitter, and not any of the first, second, or third emitters,

The method further comprises:
a first imaging event after irradiation with radiation of the first excitation wavelength providing a first luminescent detection pattern at the photodetector;
a second imaging event after irradiation with radiation of the second excitation wavelength providing a second luminescent detection pattern at the photodetector;
a third imaging event after irradiation with radiation of the third excitation wavelength providing a third luminescent detection pattern at the photodetector; and
a fourth imaging event after irradiation with radiation of the fourth excitation wavelength providing a fourth luminescent detection pattern at the photodetector.
   The combination of first, second, third, and fourth luminescent detection patterns is used identify the nucleotide that is incorporated into the polynucleotide.

In some embodiments, the method further comprises a second photodetector having a second detection window comprising a second range of wavelengths. The second range of wavelengths may be different to the first range of wavelengths of the first detection window of the first photodetector.

Optionally, in these embodiments, the method further comprises the use of third and fourth emitters, and thus four different nucleotides can be identified and distinguished using first, second, third, and fourth emitters, having emissions that can be detected using two different photodetectors.

Optionally, in these embodiments, the third and fourth emitters have excitation properties such that when irradiated with radiation of a third excitation wavelength, the peak intensity of emission of the fourth emitter in the second detection window is ≤20%, preferably ≤10%, of the peak intensity of emission of the third emitter in the second detection window, and when irradiated with radiation of a fourth excitation wavelength, the peak intensity of emission of the third emitter in the second detection window is ≤20%, preferably ≤10%, of the peak intensity of emission of the fourth emitter in the second detection window.

Optionally, in these embodiments, the third and fourth emitters have excitation properties such that when irradiated with radiation of the third excitation wavelength, the peak intensity of emission of the fourth emitter in the second detection window is ≤5%, preferably ≤2%, of the peak intensity of emission of the third emitter in the second detection window, and when irradiated with radiation of the fourth excitation wavelength, the peak intensity of emission of the third emitter in the second detection window is ≤5%, preferably ≤2%, of the peak intensity of emission of the fourth emitter in the second detection window.

Optionally, the method further comprises third and fourth imaging events.

Optionally, the second detection window of the second photodetector is the same in both the third and fourth imaging events.

Optionally, the third imaging event comprises the provision of radiation of the third excitation wavelength and the fourth imaging event comprises the provision of radiation of the fourth excitation wavelength.

Optionally, the third imaging event provides a third luminescent detection pattern, at the second photodetector, and the fourth imaging event provides a fourth luminescent detection pattern, at the second photodetector. The combination of third and fourth luminescent detection patterns is used identify the nucleotide that is incorporated into the polynucleotide.

Optionally, at a specific detection wavelength within the detection window, when irradiated with radiation of the third excitation wavelength, the brightness of the third emitter is at least 10× greater than the brightness of the fourth emitter. In some embodiments, when irradiated with radiation of the third excitation wavelength, the brightness at the specific detection wavelength of the third emitter is at least 20×, 50×, or 100× greater than the brightness of the fourth emitter.

Optionally, at a specific wavelength within the detection window, when irradiated with radiation of the fourth excitation wavelength, the brightness of the fourth emitter is at least 10× greater than the brightness of the third emitter. In some embodiments, when irradiated with radiation of the fourth excitation wavelength, the brightness at the specific detection wavelength of the fourth emitter is at least 20×, 50×, or 100× greater than the brightness of the third emitter.

Optionally, the third and/or fourth excitation wavelength may be 320 nm, 355 nm, 405 nm, 449 nm, 488 nm, 520 nm, 530 nm, 532 nm, 561 nm, 633 nm 635 nm, 640 nm, 650 nm, 660 nm, or 780 nm (in each case, each of these values may be varied by +/−2 nm, 5 nm, or 10 nm). Thus, a plurality of these excitation wavelengths may be used in combination as the third and fourth excitation wavelengths. Optionally, a plurality of these excitation wavelengths may be used in combination as any two or more of the first, second, third, and fourth excitation wavelengths.

Optionally, the third and fourth excitation wavelengths differ by at least 50 nm. In some embodiments, the third and fourth excitation wavelengths differ by at least 70 nm, 100 nm, or 120 nm.

Optionally, the photodetector may comprise a long pass filter or a band pass filter. Preferably, in embodiments comprising a long pass filter, the wavelength detection is window may be arranged to detect all wavelengths less than a specified upper limit, which may be, for example 570 nm.

Preferably, in embodiments comprising a band pass filter, the wavelength detection window has a width of ≤60 nm, such as ≤50 nm or ≤40 nm. In some embodiments, the wavelength detection window has a width of ≤30 nm.

Optionally, at least one of the third and fourth emitters has an emission peak which at 50% peak intensity overlaps by at least 5 nm with the second detection window, such as by at least, 10 nm, 15 nm, or 20 nm.

Optionally, at least one of the third and fourth emitters has an emission peak which at 50% peak intensity encompasses the width of the second detection window.

Optionally, at least one of the third and fourth emitters has an emission spectrum which overlaps with the second detection window.

Optionally, at least one of the third and fourth emitters has an emission spectrum and at least 20% of the integrated intensity of the emission spectrum is within the second detection window. For example, in some embodiments, at least 30%, 40%, 50%, 60%, 70%, 80%, or 90% of the integrated intensity of the emission spectrum is within the second detection window.

Optionally, at least one of the third and fourth emitters has an emission peak, measured in terms of the FWHM, which is entirely within the detection window.

Optionally, the difference in absorption peaks of the third emitter and the fourth emitter is at least 50 nm, such as at least 70 nm, 100 nm, or 120 nm.

Optionally, in these embodiments, when excited (i.e. when the relevant excitation wavelength is applied):
is the first nucleotide comprises only the first emitter, and not any of the second, third, or fourth emitters;
the second nucleotide comprises only the second emitter, and not any of the first, third, or fourth emitters;
the third nucleotide comprises only the third emitter, and not any of the first, second, or fourth emitters; and
the fourth nucleotide comprises only the fourth emitter, and not any of the first, second, and third emitters.

Optionally, in these embodiments, the method further comprises a third imaging event after a second application of the first excitation wavelength providing a third luminescent detection pattern (detected at the second photodetector); and a fourth imaging event after a second application of the second excitation wavelength providing a fourth luminescent detection pattern (detected at the second photodetector).

Optionally, in these embodiments, the combination of first, second, third, and fourth luminescent detection patterns is used identify the nucleotide that is incorporated into the polynucleotide.

Optionally, a positive signal in the first but not the second, third, or fourth luminescence detection pattern is indicative of the incorporation of the first nucleotide into the polynucleotide; a positive signal in the second but not the first, third, or fourth luminescence detection pattern is indicative of the incorporation of the second nucleotide into the polynucleotide; a positive signal in the third but not the first, second, or fourth luminescence detection pattern is indicative of the incorporation of the third nucleotide into the polynucleotide; and a positive signal in the fourth but not the first, second, or third luminescence detection pattern is indicative of the incorporation of the fourth nucleotide into the polynucleotide.

Optionally, the disclosed methods comprise a plurality of imaging events, and the nucleotides and associated emitters are identical and are not chemically or physically modified, added, removed, replaced, or masked between imaging events.

Optionally, any one or more of the first, second, third, and/or fourth emitters comprises a particulate emitter.

Optionally, any one or more of the first, second, third, and/or fourth emitters is dissolved in the sample.

Accordingly, in some embodiments there is provided a method for determining the sequence of a polynucleotide. The method comprises detecting in a sequencing reaction the incorporation of first, second, third, or fourth nucleotides into the polynucleotide. The method comprises the use of first and second excitation wavelengths and a photodetector having a wavelength detection window. The first nucleotide comprises a first emitter, the second nucleotide comprises a second emitter, the third nucleotide comprises first and second emitters, and the fourth nucleotide comprises neither the first nor second emitter. At the first excitation wavelength, the peak intensity of emission of the second emitter in the detection window is ≤20%, preferably ≤10%, of the peak intensity of emission of the first emitter. At the second excitation wavelength, the peak intensity of emission of the first emitter in the detection window is ≤20%, preferably ≤10%, of the peak intensity of emission of the second emitter.

In some embodiments there is provided a composition for use in a method of sequencing a nucleic acid. The composition comprises:
a first species of nucleotide comprising a first emitter;
a second species of nucleotide comprising a second emitter;
a third species of nucleotide comprising both the first and second emitters; and
a fourth species of nucleotide comprising neither the first nor second emitter.
   The first emitter is excitable when irradiated with radiation of a first excitation wavelength, and the second emitter is excitable when irradiated with radiation of a second excitation wavelength. Luminescence emission from the first emitter when irradiated with radiation of the first excitation wavelength and from the second emitter when irradiated with radiation of the second excitation wavelength is detectable at a single detection wavelength. When irradiated with radiation of the first excitation wavelength, the peak intensity of emission of the second emitter at the detection wavelength is ≤20%, preferably ≤10%, of the peak intensity of emission of the first emitter. When irradiated with radiation of the second excitation wavelength, the peak intensity of emission of the first emitter at the detection wavelength is ≤20%, preferably ≤10%, of the peak intensity of emission of the second emitter.

### DESCRIPTION OF THE DRAWINGS

The invention will now be described in more detail with reference to the drawings wherein:

FIG. 1 is a graph showing the normalised intensities of the absorption and emission spectra of ATTO490LS and Alexa Fluor 594, and detection of both dyes using a 630/30 filter.

FIG. 2 shows two graphs indicating the normalised intensities of the absorption and emission spectra of two small molecule dyes, Cascade Yellow and Alexa Fluor 532, and detection of both dyes using a 550/30 filter.

FIG. 3 shows two graphs indicating the normalised intensities of the absorption and emission spectra of a polymeric dye, Brilliant Violet 510, and a small molecule dye, Alexa Fluor 488, and detection of both dyes using a 520/15 filter.

FIG. 4 shows two graphs indicating the normalised intensities of the absorption and emission spectra of two polymeric dyes, Brilliant Violet 510 and Brilliant Blue 515, and detection of both dyes using a 515/15 filter.

FIG. 5 shows four graphs indicating the normalised intensities of the absorption and emission spectra of four emitters, Brilliant Violet 650, Alexa Fluor 633, Cascade Yellow and Alexa Fluor 532. The detection of Brilliant Violet 650 and Alexa Fluor 633 is shown using a 660/40 detection filter, and the detection of Cascade Yellow and Alexa Fluor 532 is shown using a 530/30 detection filter.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other methods, not necessarily the methods described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the disclosed methods and systems may vary considerably in their specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The disclosed methods may comprise sequencing template fragments derived from a target nucleic acid.

Unless defined otherwise, all technical and scientific terms used herein have the same meaning as is commonly understood by one of ordinary skill in the art. For clarity, the following specific terms have the specified meanings.

The term “nucleic acid” can refer to at least two nucleotide monomers linked together. Examples include, but are not limited to DNA, such as genomic or cDNA; RNA, such as mRNA, sRNA or rRNA; or a hybrid of DNA and RNA. Thus, a “nucleic acid” is a polynucleotide, such as DNA, RNA, or any combination thereof, that can be acted upon by a polymerizing enzyme during nucleic acid synthesis. The term “nucleic acid” includes single-, double-, or multiple-stranded DNA, RNA and analogs (derivatives) thereof. As apparent from the disclosure below and elsewhere herein, a nucleic acid can have a naturally occurring nucleic acid structure or a non-naturally occurring nucleic acid analog structure. A nucleic acid can contain phosphodiester bonds; however, in some embodiments, nucleic acids may have other types of backbones, comprising, for example, phosphoramide, phosphorothioate, phosphorodithioate, O-methylphosphoroamidite and peptide nucleic acid backbones and linkages. Nucleic acids can have positive backbones; non-ionic backbones, and non-ribose based backbones. Nucleic acids may also contain one or more carbocyclic sugars. The nucleic acids used in methods or compositions herein may be single stranded or, alternatively double stranded, as specified. In some embodiments a nucleic acid can contain portions of both double stranded and single stranded sequence, for example, as demonstrated by forked adapters. A nucleic acid can contain any combination of deoxyribo- and ribo-nucleotides, and any combination of bases, including uracil, adenine, thymine, cytosine, guanine, inosine, xanthanine, hypoxanthanine, isocytosine, isoguanine, and base analogs such as nitropyrrole (including 3-nitropyrrole) and nitroindole (including 5-nitroindole), etc.

A “template nucleic acid” is a nucleic acid to be detected or sequenced using any sequencing method disclosed herein. As used herein, a “primed template nucleic acid” (or alternatively, “primed template nucleic acid molecule”) is a template nucleic acid primed with (i.e., hybridized to) a primer, wherein the primer is an oligonucleotide having a 3′-end with a sequence complementary to a portion of the template nucleic acid. The primer can optionally have a free 5′-end (e.g., a portion of the primer being non-hybridized with the template), be fully hybridized to the template or can be continuous with the template (e.g., via a hairpin structure). The primed template nucleic acid includes the complementary primer and the template nucleic acid to which it is bound. Unless explicitly stated, a primed template nucleic acid can have either a 3′-end that is extendible by a polymerase, or a 3′-end that is blocked from extension. In preferred embodiments, genomic DNA fragments, or amplified copies thereof, are used as the target nucleic acid. In other preferred embodiments, mitochondrial or chloroplast DNA is used. Other embodiments are targeted to RNA or derivatives thereof such as mRNA or cDNA.

The term “nucleotide sequence” is intended to refer to the order and type of nucleotide monomers in a nucleic acid polymer. A nucleotide sequence is a characteristic of a nucleic acid molecule and can be represented in any of a variety of formats including, for example, a depiction, image, electronic medium, series of symbols, series of numbers, series of letters, series of colors, etc. A series of “A,” “T,” “G,” and “C” is letters is a well-known sequence representation for DNA that can be correlated, at single nucleotide resolution, with the actual sequence of a DNA molecule. A similar representation is used for RNA except that “T” is replaced with “U” in the series.

A “nucleotide” is a molecule that includes a nitrogenous base, a five-carbon sugar (ribose or deoxyribose), and at least one phosphate group. The term embraces, but is not limited to, ribonucleotides, deoxyribonucleotides, nucleotides modified to include exogenous labels or reversible terminators, and nucleotide analogs. The test nucleotide is preferably a native nucleotide. A “native” nucleotide refers to a naturally occurring nucleotide that does not include an exogenous label (e.g., a luminescent dye, or other label) or chemical modification such as may characterize a nucleotide analog. The term “dNTP” refers to any deoxyribonucleotide triphosphate, and a dNTP for use in the disclosed method may comprise a native nucleotide. Examples of native nucleotides that may be used as a test nucleotide in the disclosed methods include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate). The test nucleotide may be nucleotide analog. A “nucleotide analog” has one or more modifications, such as chemical moieties, which replace, remove and/or modify any of the components (e.g., nitrogenous base, five-carbon sugar, or phosphate group(s)) of a native nucleotide. Nucleotide analogs may be either incorporable or non-incorporable by a polymerase in a nucleic acid polymerization reaction. Optionally, the 3′-OH group of a nucleotide analog is modified with a moiety. The moiety may be a 3′ reversible or irreversible terminator of polymerase extension. The base of a nucleotide may be any of adenine, cytosine, guanine, thymine, or uracil, or analogs thereof. Optionally, a nucleotide has an inosine, xanthine, hypoxanthine, isocytosine, isoguanine, nitropyrrole (including 3-nitropyrrole) or nitroindole (including 5-nitroindole) base. Nucleotides may include, but are not limited to, ATP, UTP, CTP, GTP, ADP, UDP, CDP, GDP, AMP, UMP, CMP, GMP, dATP, dTTP, dUTP, dCTP, dGTP, dADP, dTDP, dCDP, dGDP, dAMP, dTMP, dCMP, and dGMP. Nucleotides may also contain terminating inhibitors of DNA polymerase, dideoxynucleotides or 2′,3′ dideoxynucleotides, which are abbreviated as ddNTPs (ddGTP, ddATP, ddTTP, ddUTP and ddCTP).

The “next correct nucleotide” (also referred to as the “cognate” nucleotide) refers to the nucleotide type that will bind and/or incorporate at the 3′ end of a primer to complement a base in a template strand to which the primer is hybridized. The base in the template strand is referred to as the “next template nucleotide” and is immediately 5′ of the base in the template that is hybridized to the 3′ end of the primer. The next correct nucleotide can be, but need not necessarily be, capable of being incorporated at the 3′ end of the primer or 3′ end of the nascent growing strand. A nucleotide having a base that is not complementary to the next template base is referred to as an “incorrect” (or “non-cognate”) nucleotide. A “marked nucleotide” refers to a nucleotide conjugated to any marker (e.g. a fluorophore) by a linker, wherein the nucleotide may or may not be incorporated into the primed strand.

The terms “label” and “marker” may be used interchangeably to refer to any group or moiety that may be used to identify, detect, and/or distinguish between nucleotides. A label may be a luminescent label, in which case, it may be referred to as an “emitter”.

A “polymerase” refers to any nucleic acid synthesizing enzyme, including but not limited to, DNA polymerase, RNA polymerase, reverse transcriptase, primase and transferase. Typically, the polymerase includes one or more active sites at which nucleotide binding and/or catalysis of nucleotide polymerization may occur. The polymerase may catalyze the polymerization of nucleotides to the 3′-end of a primer bound to its complementary nucleic acid strand. For example, a polymerase can catalyze the addition of a next correct nucleotide to the 3′ oxygen of the primer via a phosphodiester bond, thereby chemically incorporating the nucleotide into the primer.

The term “providing”, as used, for example, in relation to a test nucleotide, a marked nucleotide, a template, a primer, or a primed template nucleic acid, refers to the preparation and delivery of one or many of the relevant reagents, for example to a reaction mixture or reaction chamber.

The term “contacting” refers to the mixing together of reagents (e.g., mixing a primed template nucleic acid molecule with a reaction mixture that comprises a polymerase and the test nucleotide) so that a physical binding reaction or a chemical reaction may take place.

The term, “incorporating” or “chemically incorporating” refers to the inclusion of the cognate nucleotide, for example, by correct base pairing with the corresponding base in the template strand, or by attachment to the primer by formation of a phosphodiester bond. Accordingly, the term “incorporating” refers to the process of joining a nucleotide to the 3′-end of a primer or nascent strand by formation of a phosphodiester bond. Thus, the incorporation of a nucleotide at the 3′ end of the primer or nascent strand leads to extension of the primer or nascent strand. The incorporated nucleotide thereby provides the 3′ end of the primer or nascent strand in the subsequent sequencing cycle. The 3′ end of the primer or nascent strand thereby advances by one position along the template strand in each sequencing cycle. The terms “primer” and “nascent strand” may be interchangeably to refer to an oligonucleotide having a 3′-end with a sequence complementary to a portion of the template nucleic acid.

As used herein, “extension” refers to the process in a polymerase enzyme catalyzes addition of one or more nucleotides at the 3′-end of the primer or nascent strand, thereby leading to extension of the primer or nascent strand.

In some embodiments, the sequencing method may comprise sequencing-by-synthesis (SBS) method. In some embodiments, a SBS method may comprise four steps:
1. library preparation;
2. cluster generation;
3. sequencing; and
4. data analysis.

1. Library Preparation

Library preparation is a molecular biology protocol that converts a nucleic acid template, such as a genomic DNA sample, or cDNA sample, into a sequencing library, which can then be sequenced, for example, using a Next Generation Sequencing (NGS) instrument.

A target nucleic acid sample can, in some embodiments, be processed prior to performing other modifications. For example, a target nucleic acid sample can be amplified prior to attaching to a bead, or prior to attaching to the surface of a solid support.

Amplification is particularly useful when samples are in low abundance or when small amounts of a target nucleic acid are provided. Methods that amplify the vast majority of sequences in a genome are referred to as “whole genome amplification” methods. Examples of such methods include multiple displacement amplification (MDA), strand displacement amplification (SDA), or hyperbranched strand displacement amplification, each of which can be carried out using degenerate primers. Particularly useful methods are those that are used during sample preparation methods recommended by commercial providers of whole genome sequencing platforms (e.g. Illumina Inc., San Diego and Life Technologies Inc., Carlsbad).

The sequencing library may be prepared by random fragmentation of the nucleic acid sample. The term “fragment,” when used in reference to a first nucleic acid, is intended to mean a second nucleic acid consisting of a part or portion of the sequence of the first nucleic acid.

In some embodiments, fragmentation inherently results from amplification, for example, in cases where the portion of the template that occurs between sites where flanking primers hybridize is selectively copied.

In other embodiments, fragmentation may be achieved using chemical, enzymatic or physical techniques known in the art.

Fragments in a desired size range can be obtained using separation methods known in the art such as gel electrophoresis. Fragmentation can be carried out to obtain template nucleic acid fragments that have a minimum size of at least about 0.1 kb, 0.5 kb, 1 kb, 2 kb, 3, kb, 4 kb, 5 kb, 10 kb or longer in length.

Adapters, which may be referred to as “library adapters” may be ligated to the template fragments, such as, for example, ligation of 5′ and 3′ adapters to each DNA fragment. “Tagmentation” may be used to combine the fragmentation and ligation reactions into a single step that may increase the efficiency of the library preparation process.

Adapter-ligated fragments may be amplified and purified by any suitable method currently used in the art. For example, adapter-ligated fragments may be PCR amplified and gel purified.

The fragments that are produced from one or more nucleic acid templates can be captured randomly at locations on a solid support surface.

Solid supports can be two-or three-dimensional and can be a planar surface (e.g., a glass slide) or can be shaped. Useful materials include glass (e.g., controlled pore glass (CPG)), quartz, plastic (such as polystyrene (low cross-linked and high cross-linked polystyrene), polycarbonate, polypropylene and poly(methylmethacrylate)), acrylic copolymer, polyamide, silicon, metal (e.g., alkanethiolate-derivatized gold), cellulose, nylon, latex, dextran, gel matrix (e.g., silica gel), polyacrolein, or composites. Suitable three-dimensional solid supports include, for example, spheres, microparticles, beads, membranes, slides, plates, micromachined chips, tubes (e.g., capillary tubes), microwells, microfluidic devices, channels, filters, or any other structure suitable for anchoring a nucleic acid. Solid supports can include planar microarrays or matrices capable of having regions that include populations of nucleic acids or primers.

Examples include nucleoside-derivatized CPG and polystyrene slides; derivatized magnetic slides; polystyrene grafted with polyethylene glycol, and the like.

A solid support to which nucleic acids may be attached in the sequencing method have a continuous or monolithic surface. Thus, fragments can attach at spatially random locations wherein the distance between nearest neighbor fragments (or nearest neighbor clusters derived from the fragments) may be variable. The resulting arrays may have a variable or random spatial pattern of features.

Different template fragments that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features or locations on the same substrate. Exemplary sites include, for example, wells in a substrate, beads (or other particles) in or on a substrate, projections from a substrate, ridges on a substrate or channels in a substrate. The sites of an array can be separate substrates each bearing a different molecule. Exemplary arrays in which separate substrates are located on a surface include, for example, those having beads in wells.

The disclosed methods can advantageously use arrays having a high density of features such as, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², 10⁷ features/cm², 5×10⁷ features/cm², 10⁸ features/cm², 5×10⁸ features/cm², 10⁹ features/cm², 5×10⁹ features/cm², or higher.

Flow cells provide a convenient format for housing an array of nucleic acid fragments for use in the disclosed methods. As used herein, the term “flow cell” is intended to mean a chamber having a surface across which one or more fluid reagents can be flowed. Generally, a flow cell will have an ingress opening and an egress opening to facilitate flow of fluid. Flow cells provide a convenient format for use in the disclosed method that involves repeated delivery of reagents in cycles. For example, to initiate a first SBS cycle, one or more dNTPs, DNA polymerase, etc., can be flowed into/through a flow cell that houses an array of nucleic acid fragments. Washes can easily be carried out in the flow cell between the various delivery steps. The cycle can be repeated n times to extend the primer by n nucleotides, thereby detecting a sequence of length n.

For cluster generation, the library of adapter-ligated template fragments may be loaded into a flow cell where fragments are captured on a lawn of surface-bound binding molecules, such as oligonucleotides complementary to the library adapters.

DNA nanoballs can also be used in the disclosed methods and methods for preparing and using DNA nanoballs for genomic sequencing are known in the art. Briefly, following genomic DNA fragmentation consecutive rounds of adaptor ligation, amplification and digestion results in head to tail concatamers of multiple copies of the circular genomic DNA template/adaptor sequences which are circularized into single stranded DNA by ligation with a circle ligase and rolling circle amplified. The adaptor structure of the concatamers promotes coiling of the single stranded DNA thereby creating compact DNA nanoballs. The DNA nanoballs can be captured on substrates, preferably to create an ordered or patterned array such that distance between each nanoball is maintained thereby allowing sequencing of the separate DNA nanoballs.

Sequencing utilizing the methods and compositions described herein can also be performed in a microtiter plate, for example in high density reaction plates or slides. For example, genomic targets can be prepared by emPCR technologies. Reaction plates or slides can be created from fiber optic material capable of capturing and recording light generated from a reaction, for example from a luminescent reaction. The core material can be etched to provide discrete reaction wells capable of holding at least one emPCR reaction bead. Such slides/plates can contain over a 1.6 million wells. The created slides/plates can be loaded with the target sequencing reaction emPCR beads and mounted to an instrument where the sequencing reagents are provided and sequencing occurs.

2. Cluster Generation

Cluster generation is a process of clonal amplification of target nucleic acid templates which may be used or required, for example, for imaging systems which cannot detect single luminescence events. Various suitable methods for clonally amplifying nucleic acid template molecules to produce clusters of cloned template will be known to the skilled person. Any suitable method may be used, and typically, these methods comprise polymerase chain reaction (PCR)-based techniques. The cluster generation procedure is relatively complicated and time-consuming, and may introduce errors into the cloned template nucleic acids.

In the cluster generation step, each template fragment is clonally amplified into distinct clusters. The result is a clonal grouping of identical template fragments bound to the surface of the flow cell. Each cluster on the flow cell produces a single sequencing read. For example, 10,000 clusters on the flow cell would produce 10,000 sequence reads. Where paired-end reads are implemented, a second read of each sequence would also be performed.

Each cluster is seeded by a single template nucleic acid fragment and is clonally amplified, for example, using a PCR-based approach, such as involving the use of forward and reverse primers that are attached to the support within the flow cell. In current sequencing methods, a typical cluster has in the order of 1000 copies.

To enable the generation of defined clusters, the template fragments may be captured onto surfaces that are patterned for example, with embedded beads (typically 1-2 μm in diameter) or wells (typically 200-600 nm in diameter). Each bead or well only captures a single template fragment and the size of the bead or well defines the maximum size of the cluster. The structured organization provided by the patterned surface of the flow cell provides improved, regular spacing of template clusters, and increased cluster density, which provides advantages over non-pattered clusters, such as in relation to signal detection.

Bridge amplification may be used to generate clusters. Bridge amplification may occur on the surface of the flow cell. For example, in currently used methods, the surface of the flow cell is coated with a “lawn” of oligonucleotides. In the first step of bridge amplification, a single-stranded sequencing library (with complementary adapter ends) is loaded into the flow cell. Individual molecules in the library bind to complementary oligos as they “flow” across the oligo lawn. Priming occurs as the opposite end of a ligated fragment bends over and “bridges” to another complementary oligo on the surface. Repeated denaturation and extension cycles (similar to PCR) result in localized amplification of single molecules into millions of unique, clonal clusters across the flow cell.

Other suitable amplification methods known in the art can also be used to produce immobilized amplicons from immobilized nucleic acid fragments. For example one or more clusters can be formed via solid-phase PCR, solid-phase MDA, solid-phase RCA etc., whether one or both primers of each pair of amplification primers are immobilized.

3. Sequencing

In some embodiments, the disclosed method comprises the detection of single nucleotides as they are incorporated at the 3′ end of the primer or 3′ end of the nascent growing strand.

In some embodiments, nucleotides are added to a nucleic acid primer thereby extending the primer in a template-dependent manner. Detection of the order and type of nucleotides added to the primer can be used to determine the sequence of the template.

The nucleotides are labelled, such as with a luminescent marker, that may be used to detect and identify the nucleotide.

During each sequencing cycle, a single labeled nucleotide, which is the next complementary nucleotide (i.e. comprises a base complementary to the next base of the template strand), is incorporated into the nucleic acid chain in a template-dependent manner due to complementary hydrogen bonding with the corresponding nucleotide in the template fragment.

A polymerase enzyme may subsequently catalyze the chemical addition of the next complementary nucleotide into the nucleic acid chain.

In each sequencing cycle, the next complementary nucleotide is identified by excitation of the emitter, or light emitting marker, and detection of the emitted luminescence, for example, using laser excitation and imaging.

A plurality of different species of test nucleotide may be included in the reaction mixture in a single sequencing cycle. Thus, labels associated with each different nucleotide species may be distinguishable and identifiable, for example on the basis of different luminescence emission spectra. In these embodiments, the luminescence emission that is detected is used to determine which of the different species of test nucleotides is incorporated into the primed strand and, thus represents the next complementary nucleotide.

In some embodiments, one or more of the nucleotides may be pre-labelled with the relevant marker prior to inclusion in the reaction mixture and/or prior to incorporation into the growing strand of the nucleic acid that is being sequenced. In other embodiments, one or more of the nucleotides may be labelled after correctly base pairing with the relevant nucleotide in the template strand, and/or after incorporation into the growing strand. For example, one of more of the nucleotides may comprise a moiety, such as a linker, to which a detectable label may be directly or indirectly attached to thereby detect and identify the nucleotide.

In any event, at the point of detection, such as for example, when electromagnetic radiation at an excitation wavelength is applied to an emitter, the nucleotide that is being detected comprises the label, in the sense that the label is associated with the nucleotide in such a way that it may be used to specifically detect and identify the nucleotide.

In some sequencing methods, each nucleotide is associated with a different luminescent label that may be used to detect and identify the nucleotide. Thus, the number of possible different nucleotides to be detected is equal to the number of different labels used, and the number of detection channels required.

In some embodiments of the disclosed method, however, the number of possible different nucleotides is greater than the number of different labels used, and also the number of detection channels required.

In all embodiments of the disclosed method, the number of possible different nucleotides that may be detected is greater than the number detection channels or “detection filters” used to detect them.

Thus, as discussed in more detail below, the disclosed methods advantageously provide a reduction in the number of detection channels required to distinguish all of the possible different nucleotides in a sequencing process.

The nucleotide may comprise a “terminator” which may be a “reversible terminator”. A nucleotide having a terminator or reversible terminator moiety can be used such that subsequent extension cannot occur until a deblocking agent is delivered to remove the terminator moiety. Thus, after nucleotide incorporation and identification, the terminator may be removed, such as by enzymatic cleavage, to allow the next sequencing cycle to commence.

The linker attaching the label to the nucleotide in the disclosed method may be cleavable or otherwise arranged to allow dissociation of the label from the nucleotide. Thus, after incorporation and identification of the test nucleotide, the emitter or other label may be removed to allow the next sequencing cycle to commence and the next complementary nucleotide to be identified. The result is base-by-base sequencing of the template fragment nucleic acids.

The term “linker” is intended to mean a chemical bond or moiety that bridges two moieties, for example by covalent linkage or the formation of a stable complex. A linker can be, for example, the sugar-phosphate backbone that connects nucleotides in a nucleic acid moiety. In the disclosed method, a linker may be used to conjugate a test nucleotide to a label such as a fluorophore or other emitter. The linker can include, for example, one or more of a nucleotide moiety, a nucleic acid moiety, a non-nucleotide chemical moiety, a nucleotide analogue moiety, amino acid moiety, polypeptide moiety, or protein moiety.

The terms “cleave”, “cleavage site”, and similar terms, refer to a moiety in a molecule, such as a linker, that can be modified or removed to physically separate two other moieties of the molecule.

In some embodiments of the disclosed method, a linker may be used to associate the test nucleotide to a terminator. The linker may be a cleavable linker, such that at the appropriate point in the sequencing cycle the linker is cleaved, thereby removing the terminator from the nucleotide. In some embodiments of the disclosed method, the linker that is used to associate the test nucleotide and terminator comprises the same type of cleavable linkage that is present in the linker conjugating the test nucleotide to the fluorophore or other detectable label. Thus, at the appropriate point in the sequencing cycle, a single agent, such as a single type of cleaving enzyme, may be used to remove both the terminator and label from the test nucleotide. In some embodiments of the disclosed method, a single linker may be arranged to conjugate both the terminator and label to the test nucleotide. In some embodiments of the disclosed method, the linker that is used to associate the test nucleotide and terminator may comprise a different type of cleavable linkage to that present in the linker conjugating the test nucleotide to the label.

In some embodiments, a plurality of different nucleic acid fragments can be sequenced simultaneously under conditions where events occurring for different templates can be distinguished, for example due to being present at different locations in an array.

4. Data Analysis

During data analysis, the newly identified sequence reads of the template fragment are aligned, and the target nucleic acid sequence may thus be determined.

Following alignment, many variations of analysis are possible, including single nucleotide polymorphism (SNP), insertion-deletion (indel) identification, and read counting for RNA methods, phylogenetic or metagenomic analysis.

The skilled person will appreciate that the disclosed methods are not limited to sequencing-by-synthesis methods, and may be applied to various other methods involving the use of luminescence to identify a nucleotide. Such methods include, for example, methods for the analysis of short tandem repeat (STR) markers, single nucleotide polymorphisms (SNPs), methylation patterns, ChIP analysis, and RNA transcription. Also included are methods of analysis of any nucleic acid template, including, for example, DNA from any organism or mixed population such as a microbiome, whole genomes, RNA transcripts for expression analysis, cancer samples (such as methods of analysing somatic variants and/or tumour subclones), and mitochondrial DNA.

### Detection Methods

The present disclosure provides methods for determining the identity of an incorporated nucleotide, from a number of different possible nucleotides, in a nucleic acid sequencing cycle. The method involves the use of radiation of first and second excitation wavelengths and a photodetector having a detection window comprising a range of wavelengths. At least a first nucleotide, but not a second nucleotide, which is of a different species to the first nucleotide, comprises a first emitter. At least the second nucleotide, but not the first nucleotide, comprises a second emitter, which has different excitation properties to the first emitter. The first and second emitters have excitation properties such that when irradiated with radiation of the first excitation wavelength, the peak intensity of emission of the second emitter in the detection window is ≤20%, preferably ≤10%, of the peak intensity of emission of the first emitter. When irradiated with radiation of the second excitation wavelength, the peak intensity of emission of the first emitter in the detection window is ≤20%, preferably ≤10%, of the peak intensity of emission of the second emitter. The first and second nucleotides each comprise the relevant emitter at the point at which the emitters are excited, and the emission (or absence of emission) from the emitter in response to irradiation with radiation of the first and second excitation wavelengths may thereby be used to identify and distinguish the first and second nucleotides.

One advantage of this method is that all of the possible different nucleotides in the sequencing reaction may be distinguished and identified using a number of detection windows that is fewer than the number of different nucleotides being detected. This leads to a number of advantages in terms of processing time and efficiency, as discussed below.

There are several embodiments of the disclosed method comprising this principle, in which at least first and second emitters are excited using radiation of first and second excitation wavelengths and detected using the same photodetector, and these different embodiments may all be employed to identify and distinguish all four of the nucleotides present in a sequencing reaction using only one or two photodetectors.

In some embodiments, a single photodetector may be used to detect the emission from two, three, or four different emitters. The different emitters have different excitation properties such that they are excited by radiation of different excitation wavelengths, and when excited can be detected using a single photodetector.

### Embodiments Using a Single Photodetector and Two Emitters

Thus, in some embodiments, as discussed in more detail below, four different nucleotides can be identified and distinguished using only first and second emitters having different excitation properties, and emissions that can be detected using a single photodetector.

Preferably, the first nucleotide comprises the first emitter but not the second emitter; the second nucleotide comprises the second emitter but not the first emitter; the third nucleotide comprises both the first and second emitters; and the fourth nucleotide comprises neither the first nor second emitter.

Preferably, the method further comprises a first imaging event after application of radiation of the first excitation wavelength providing a first luminescent detection pattern at the photodetector, and a second imaging event after application of radiation of the second excitation wavelength providing a second luminescent detection pattern at the photodetector. The combination of first and second luminescent detection patterns is used identify the nucleotide that is incorporated into the polynucleotide.

Preferably in these methods, a positive signal in the first but not the second luminescence detection pattern is indicative of the incorporation of the first nucleotide into the polynucleotide; a positive signal in the second but not the first luminescence detection pattern is indicative of the incorporation of the second nucleotide into the polynucleotide; a positive signal in both the first and second luminescence detection patterns is indicative of the incorporation of the third nucleotide into the polynucleotide; and a positive signal in neither the first nor second luminescence detection pattern is indicative of the incorporation of the fourth nucleotide into the polynucleotide.

### Embodiments Using a Single Photodetector and Three Emitters

In some embodiments, four different nucleotides can be identified and distinguished using only first, second, and third emitters having different excitation properties, and emissions that can be detected in a single photodetector.

Preferably, the first nucleotide comprises the first emitter but not the second or third emitter; the second nucleotide comprises the second emitter but not the first or third emitter; the third nucleotide comprises the third emitter but not the first or second emitters; and the fourth nucleotide comprises no emitter.

The first, second, and third emitters have excitation properties such that when irradiated with radiation of the first excitation wavelength, the peak intensity of emission of the second and third emitters in the detection window is ≤20%, preferably ≤10%, of the peak intensity of emission of the first emitter. When irradiated with radiation of the second excitation wavelength, the peak intensity of emission of the first and third emitters in the detection window is ≤20%, preferably ≤10%, of the peak intensity of emission of the second emitter. When irradiated with radiation of a third excitation wavelength, the peak intensity of emission of the first and second emitters in the detection window is ≤20%, preferably ≤10%, of the peak intensity of emission of the third emitter.

Preferably, the method further comprises: a first imaging event after irradiation with radiation of the first excitation wavelength, providing a first luminescent detection pattern at the photodetector; a second imaging event after irradiation with radiation of the second excitation wavelength, providing a second luminescent detection pattern at the photodetector; and a third imaging event after irradiation with radiation of the third excitation wavelength, providing a third luminescent detection pattern at the photodetector. The combination of first, second, and third luminescent detection patterns is used identify the nucleotide that is incorporated into the polynucleotide.

Preferably in these methods, a positive signal in the first but not the second or third luminescence detection pattern is indicative of the incorporation of the first nucleotide into the polynucleotide; a positive signal in the second but not the first or third luminescence detection pattern is indicative of the incorporation of the second nucleotide into the polynucleotide; a positive signal in the third but not the first or second luminescence detection pattern is indicative of the incorporation of the third nucleotide into the polynucleotide; and a positive signal in none of the first, second, or third luminescence detection patterns is indicative of the incorporation of the fourth nucleotide into the polynucleotide.

Thus, four different nucleotides can be identified and distinguished using only three different emitters and a single photodetector.

In other arrangements, the fourth nucleotide may be identified and distinguished by a positive emission signal, rather than by the absence of a positive signal in any of the detection patterns. For example, the fourth nucleotide may be labelled using a plurality of the first, second, and/or third emitters, such as any combination of two or all three of the emitters, and in this arrangement, a positive signal in the relevant plurality of luminescence detection patterns may be indicative of the incorporation of the fourth nucleotide into the polynucleotide.

### Embodiments Using a Single Photodetector and Four Emitters

In some embodiments, four different nucleotides can be identified and distinguished using first, second, third, and fourth emitters having different excitation properties, and emissions that can be detected in a single photodetector.

Preferably, the first nucleotide comprises the first emitter but not any of the second, third, or fourth emitters; the second nucleotide comprises the second emitter but not any of the first, third, or fourth emitters; the third nucleotide comprises the third emitter but not any of the first, second, or fourth emitters; and the fourth nucleotide comprises the fourth emitter but not any of the first, second, or third emitters.

The first, second, third, and fourth emitters have excitation properties such that when irradiated with radiation of the first excitation wavelength, the peak intensity of emission of the second, third, and fourth emitters in the detection window is ≤20%, preferably ≤10%, of the peak intensity of emission of the first emitter. when irradiated with radiation of the second excitation wavelength, the peak intensity of emission of the first, third, and fourth emitters in the detection window is ≤20%, preferably ≤10%, of the peak intensity of emission of the second emitter. When irradiated with radiation of a third excitation wavelength, the peak intensity of emission of the first, second, and fourth emitters in the detection window is ≤20%, preferably ≤10%, of the peak intensity of emission of the third emitter. When irradiated with radiation of a fourth excitation wavelength, the peak intensity of emission of the first, second, and third emitters in the detection window is ≤20%, preferably ≤10%, of the peak intensity of emission of the fourth emitter.

Preferably, the method further comprises: a first imaging event after irradiation with radiation of the first excitation wavelength, providing a first luminescent detection pattern at the photodetector; a second imaging event after irradiation with radiation of the second excitation wavelength, providing a second luminescent detection pattern at the photodetector; a third imaging event after irradiation with radiation of the third excitation wavelength, providing a third luminescent detection pattern at the photodetector; and a fourth imaging event after irradiation with radiation of the fourth excitation wavelength, providing a fourth luminescent detection pattern at the photodetector. The combination of first, second, third, and fourth luminescent detection patterns is used identify the nucleotide that is incorporated into the polynucleotide.

Preferably in these methods, a positive signal in the first but not any of the second, third, or fourth luminescence detection patterns is indicative of the incorporation of the first nucleotide into the polynucleotide; a positive signal in the second but not any of the first, third, or fourth luminescence detection patterns is indicative of the incorporation of the second nucleotide into the polynucleotide; a positive signal in the third but not any of the first, second, or fourth luminescence detection patterns is indicative of the incorporation of the third nucleotide into the polynucleotide; and a positive signal in the fourth but not any of the first, second, or third luminescence detection patterns is indicative of the incorporation of the fourth nucleotide into the polynucleotide.

Thus, four different nucleotides can be identified and distinguished using four different emitters and a single photodetector.

### Methods Using Two Photodetectors

In other embodiments of the disclosed method, the method comprises the use of two detection filters, having different detection windows. Thus, in these embodiments the method comprises, in addition to the first photodetector, a second photodetector having a second detection window that is different to the first detection window, comprising a second range of wavelengths.

In some embodiments, the method comprises the use of two detection filters, having different detection windows, and three different emitters.

### Embodiments Using Two Photodetectors and Three Emitters

In some embodiments, four different nucleotides can be identified and distinguished using only first, second, and third emitters having emissions that can be detected in two different photodetectors. The emission from the first and second emitters in response to irradiation with radiation of first and second excitation wavelengths respectively can be detected in a first detection filter. The emission from the third emitter, for example, in response to irradiation with radiation of an excitation wavelength that may be for example, the first or second excitation wavelength, can be detected in a second detection filter.

Preferably, the first nucleotide comprises the first emitter but not the second or third emitter; the second nucleotide comprises the second emitter but not the first or third emitter; the third nucleotide comprises the third emitter but not the first or second emitters; and the fourth nucleotide comprises no emitter.

The first and second emitters have excitation properties such that when irradiated with radiation of the first excitation wavelength, the peak intensity of emission of the second emitter in the first detection window is ≤20%, preferably ≤10%, of the peak intensity of emission of the first emitter. When irradiated with radiation of the second excitation wavelength, the peak intensity of emission of the first emitter in the first detection window is ≤20%, preferably ≤10%, of the peak intensity of emission of the second emitter.

When irradiated with radiation of the third excitation wavelength, which may be the same as the first or second excitation wavelength, emission from the third emitter may be detected in the second detection window. Preferably, when irradiated with radiation of the excitation wavelength used to detect emission from the third emitter, the peak intensity of emission of the third emitter in the first detection window is ≤20%, preferably ≤10%, of the peak intensity of emission of the third emitter in the second detection window. Also, when irradiated with radiation of the first excitation wavelength, the peak intensity of emission of the first emitter in the second detection window is ≤20%, preferably ≤10%, of the peak intensity of emission of the first emitter in the first detection window. When irradiated with radiation of the second excitation wavelength, the peak intensity of emission of the second emitter in the second detection window is ≤20%, preferably ≤10%, of the peak intensity of emission of the second emitter in the first detection window.

Preferably, the method further comprises: a first imaging event after irradiation with radiation of the first excitation wavelength, providing a first luminescent detection pattern at the first photodetector; a second imaging event after irradiation with radiation of the second excitation wavelength, providing a second luminescent detection pattern at the first photodetector; and a third imaging event after irradiation with radiation of the third excitation wavelength, providing a third luminescent detection pattern at the second photodetector (i.e. which is the first luminescent detection pattern at the second photodetector). The combination of first, second, and third luminescent detection patterns is used identify the nucleotide that is incorporated into the polynucleotide.

Preferably in these methods, a positive signal in the first but not the second luminescence detection pattern is indicative of the incorporation of the first nucleotide into the polynucleotide; a positive signal in the second but not the first luminescence detection pattern is indicative of the incorporation of the second nucleotide into the polynucleotide; a positive signal in the third luminescence detection pattern is indicative of the incorporation of the third nucleotide into the polynucleotide; and a positive signal in none of the first, second, or third luminescence detection patterns is indicative of the incorporation of the fourth nucleotide into the polynucleotide.

Thus, four different nucleotides can be identified and distinguished using three different emitters, two different excitation wavelengths, and two different photodetectors.

### Embodiments Using Two Photodetectors and Four Emitters

In some embodiments using two photodetectors, the fourth nucleotide may be identified and distinguished by a positive emission signal, rather than by the absence of a positive signal in any of the detection patterns.

For example, in some embodiments, four different nucleotides can be identified and distinguished using first, second, third, and fourth emitters, having emissions that can be detected using two different photodetectors.

Preferably, the first nucleotide comprises the first emitter but not any of the second, third, or fourth emitters; the second nucleotide comprises the second emitter but not any of the first, third, or fourth emitters; the third nucleotide comprises the third emitter but not any of the first, second, or fourth emitters; and the fourth nucleotide comprises the fourth emitter but not any of the first, second, or third emitters.

Preferably, the first and second emitters have excitation properties such that when irradiated with radiation of the first excitation wavelength, the peak intensity of emission of the second emitter in the first detection window is ≤20%, preferably ≤10%, of the peak intensity of emission of the first emitter. When irradiated with radiation of the second excitation wavelength, the peak intensity of emission of the first emitter in the first detection window is ≤20%, preferably ≤10%, of the peak intensity of emission of the second emitter. Also, when irradiated with radiation of the first excitation wavelength, the peak intensity of emission of the first emitter in the second detection window is ≤20%, preferably ≤10%, of the peak intensity of emission of the first emitter in the first detection window. When irradiated with radiation of the second excitation wavelength, the peak intensity of emission of the second emitter in the second detection window is ≤20%, preferably ≤10%, of the peak intensity of emission of the second emitter in the first detection window.

Preferably, the third and fourth emitters have excitation properties such that when irradiated with radiation of the first excitation wavelength, the peak intensity of emission of the fourth emitter in the second detection window is ≤20%, preferably ≤10%, of the peak intensity of emission of the third emitter in the second detection window, and when irradiated with radiation of the second excitation wavelength, the peak intensity of emission of the third emitter in the second detection window is ≤20%, preferably ≤10%, of the peak intensity of emission of the fourth emitter in the second detection window. Also, when irradiated with radiation of the first excitation wavelength, the peak intensity of emission of the third emitter in the first detection window is ≤20%, preferably ≤10%, of the peak intensity of emission of the third emitter in the second detection window. When irradiated with radiation of the second excitation wavelength, the peak intensity of emission of the fourth emitter in the first detection window is ≤20%, preferably ≤10%, of the peak intensity of emission of the fourth emitter in the second detection window.

Preferably, the method further comprises: a first imaging event after irradiation with radiation of the first excitation wavelength, providing a first luminescent detection pattern (detected at the first photodetector); a second imaging event after irradiation with radiation of the second excitation wavelength, providing a second luminescent detection pattern (detected at the first photodetector); a third imaging event after a second irradiated with radiation of the first excitation wavelength, providing a third luminescent detection pattern (detected at the second photodetector); and a fourth imaging event after a second irradiation with radiation of the second excitation wavelength, providing a fourth luminescent detection pattern (detected at the second photodetector).

Preferably, the combination of first, second, third, and fourth luminescent detection patterns is used identify the nucleotide that is incorporated into the polynucleotide. Thus, a positive signal in the first but not the second, third, or fourth luminescence detection pattern is indicative of the incorporation of the first nucleotide into the polynucleotide; a positive signal in the second but not the first, third, or fourth luminescence detection pattern is indicative of the incorporation of the second nucleotide into the polynucleotide; a positive signal in the third but not the first, second, or fourth luminescence detection pattern is indicative of the incorporation of the third nucleotide into the polynucleotide; and a positive signal in the fourth but not the first, second, or third luminescence detection pattern is indicative of the incorporation of the fourth nucleotide into the polynucleotide.

Thus, four different nucleotides can be identified and distinguished using four different emitters, two different excitation wavelengths, and two different photodetectors.

The use of only one or two detection filters provides advantages over existing sequencing methods, which typically require the use of a specific detection filter for each nucleotide. The sequencing methods may be performed more quickly and economically, and on smaller formats, because fewer detection filters need to be present, relative to previous sequencing methods.

The use of a single detection filter, in particular, allows for sequencing methods to be performed more quickly and economically, because fewer detection steps need to be performed, relative to previous sequencing methods. In methods comprising the use of a single detection filter, sequencing cycles can be performed more quickly because no time is required to change detection filters, thereby increasing throughput capacity.

Thus, the disclosed methods provide advantages in terms of reduced size and cost of instrument hardware, and reduced reagent usage.

In the case of a nucleic acid sample, a data acquisition step can be performed on the sample to acquire a collection of less than four different signal types (preferably two different signal types) and yet the sequence location for all four of the different nucleotide types can be determined for the sample.

### Signal States

The disclosed methods may include, for each imaging event, correlating one or more nucleotide species to a dark state, correlating one or more nucleotide species to a signal state, correlating one or more nucleotide species to a grey state, and/or correlating one or more nucleotide species to a change in state between two imaging events (such as first and second imaging events), between a dark state, a grey state or a signal state.

References to a “positive signal” or the “detection of a positive signal” refer to a signal state and in some embodiments may also refer to a grey state. References to a “negative signal” or the “detection of a negative signal” refer to a dark state.

A “signal state,” when used in reference to a detection event, means a condition in which a specific signal is produced in the detection event. For example, a nucleotide can be in a signal state and detectable when attached to a luminescent label that is excited when irradiated with radiation of a specific excitation wavelength and detected by emission in an emission detection step in a sequencing method, using a specific detection filter.

The term “dark state,” when used in reference to a detection event, means a condition in which a specific signal is not produced in the detection event. For example, a nucleotide can be in a dark state when the nucleotide does not emit above a threshold level in an emission detection step in a sequencing method, using a single detection filter. For example, a nucleotide may lack a luminescent label, and/or may be attached to a luminescent label that is excited when irradiated with radiation of a specific excitation wavelength that is different to the excitation wavelength used in relation to that particular detection event.

Dark state detection may also include any background emission which may be present in the absence of a luminescent label that is excited when irradiated with radiation of the specific excitation wavelength being used in relation to that particular detection event. For example, some reaction components, which may include luminescent labels that are excited when irradiated with radiation of a different excitation wavelength to the specific excitation wavelength being used, may demonstrate minimal emission in response to the excitation wavelength being used. As such, there may be background emission from such components. Further, background emission may be due to light scatter, for example from adjacent sequencing reactions, which may be detected by a detector. In addition, “dark state” can include background emission produced when an emissive moiety is not specifically included, such as when a nucleotide lacking a luminescent label is used. However, such background emission is distinguishable from a signal state or a grey state and, as such, nucleotide incorporation of an unlabelled nucleotide (or “dark” nucleotide) is still discernible. Likewise, nucleotide incorporation of a nucleotide that is attached to a luminescent label that is excited when irradiated with radiation of a specific excitation wavelength that is different to the excitation wavelength used in relation to that particular detection event can also be distinguished from a signal state or a grey state.

The term “grey state,” when used in reference to a detection event, means a condition in which an attenuated signal is produced in the detection event. For example, a population of nucleotides of a particular type can be in a grey state when a first subpopulation of the nucleotides attached to a first luminescent label that is detected in a luminescence detection step of a sequencing method, while a second subpopulation of the nucleotides lacks the first luminescent label and does not give emission that is specifically detected in the luminescence detection step when irradiated with radiation of the excitation wavelength of the first luminescent label. The second subpopulation of the nucleotides, may, for example, comprise a second luminescent label that may be detected when irradiated with radiation of at an excitation wavelength that is different to the excitation wavelength of the first luminescent label, such that the first and second luminescent labels may be detected in the same detector but are distinguishable on the basis that they are excited using different excitation wavelengths.

Previous methods have been described, which involve at least one nucleotide modification step during an individual sequencing cycle. Such methods can include several cycles of nucleotide addition and the cycles can include orthogonal steps of acquiring signals from the sequencing sample, then modifying one or more nucleotides in the sequencing sample to change their state (e.g. between a signal state or a dark state or grey state), and then acquiring a second set of signals from the sequencing sample. For example, in such methods, particular nucleotide types are in a signal state due to an attached luminescent label, particular nucleotide types are in a dark state due to the absence of the label, particular nucleotides are converted from a signal state to a dark state by cleaving a linker that attaches a luminescent label and/or particular nucleotides are converted from a dark state to a signal state by binding a luminescent label to the nucleotide that did not otherwise have the label.

The disclosed methods, however, provide the ability to distinguish a number of different possible nucleotides in a nucleic sample without any requirement for the modification, addition, and/or removal of any nucleotide or associated label. Advantageously, despite the fact the disclosed methods do not require any modification, addition, and/or removal of the nucleotides between imaging events, the disclosed methods may be used to identify and distinguish between nucleotides in sequencing reaction using a number of detection filters that is smaller than the number of different possible nucleotides being detected. In particular, four different nucleotides may be distinguished using one or two different detection filters.

The disclosed methods provide for detection of multiple different nucleotides in a sequencing reaction by distinguishing between a plurality of luminescent moieties having different excitation properties, but that are capable of detection using a number of detection filters that is fewer than the number of different possible nucleotides in the sequencing reaction. Preferably, the disclosed methods use only one or two different detection filters.

### Detection Filters

Any suitable detection filter or filters known in the art may be used in the disclosed methods, and suitable detection filters will be known to the skilled person.

The detection filter may comprise a long pass filter or a band pass filter.

In embodiments involving the use of a long pass filter, the wavelength detection window may be arranged to detect all wavelengths less than a specified upper limit, which may be, for example 570 nm.

In embodiments involving the use of a band pass filter, the detection window of the detection filter may have a width of less than 100 nm, such as less than 90 nm or less than 75 nm. Preferably the width of the detection window is less than or equal to about 60 nm, such as less than or equal to about 50 nm or 40 nm. The width of the detection window of the detection filter may be, for example, less than or equal to about 30 nm.

As discussed in more detail below, the detection filter or filters for use in the disclosed methods are preferably selected based on the emission properties of the luminescent labels used in the sequencing method.

Thus, in embodiments comprising the use of a single detection filter, the detection filter may be selected in combination with the first and second luminescent labels such that both the first and second luminescent labels have emission spectra which overlap with the detection window of the selected detection filter.

In embodiments comprising the use of two detection filters, the detection filters may be selected in combination with the first, second, third, and fourth luminescent labels such that both the first and second luminescent labels have emission spectra which overlap with the detection window of the first but not the second detection filter, and the third and fourth luminescent labels have emission spectra which overlap with the detection window of the second but not the first detection filter.

Typically, a reaction cycle will be carried out by delivering all four nucleotide types to a nucleic acid sample in the presence of a polymerase, for example a DNA or RNA polymerase, during a primer extension reaction. The presence of four nucleotide types provides an advantage of increasing polymerase fidelity compared to the use of fewer than four nucleotide types. The use of a plurality of different luminescent labels that are excited when irradiated with radiation of different wavelengths, and have emission spectra that can be detected in the same detection channel allows multiple nucleotide types to be present simultaneously during a polymerase extension reaction, thereby increasing fidelity while also allowing a reduced number of detection filters, including even just a single detection filter, to be used in each cycle, which serves to provide more simplified optics. Use of simplified optics is preferential as compared to systems that rely on more complex optics to record output from multiple different labels to distinguish different nucleotide types that are present simultaneously in an extension reaction.

In some embodiments, fewer than four different types of nucleotides can be present during a polymerase extension reaction.

In some embodiments, the disclosed methods for sequencing a nucleic acid comprise the use of two or more different luminescent labels for direct or indirect detection of three different nucleotide types and one nucleotide type that is not detected by the presence of a luminescent signal (i.e. a positive signal) but is instead detected by a lack or absence of a luminescent signal (i.e. a negative signal).

In some embodiments, methods for sequencing a nucleic acid comprise the use of two or more different luminescent labels, that are excited when irradiated with radiation of different excitation wavelengths but have emission spectra that may be detected using the same detection filter, for direct or indirect detection of three different nucleotide types, and one nucleotide type that is not detected by the presence of a luminescent signal but is instead detected by a lack or absence of a luminescent signal.

The excitation and emission spectra are such that two different wavelengths of radiation are used to excite the two or more different luminescent moieties and their emitted signals pass through a single optical filter. Detection of luminescence to determine the sequence of a nucleic acid sample is performed at least twice during a sequencing reaction providing patterns of luminescence such as luminescence transitions patterns, the luminescence patterns determining the sequence of the nucleic acid target. As such, the methods described herein are time and cost efficient and allow for simplification of associated sequencing instrumentation.

The disclosed methods are not limited to nucleic acid sequencing and may also be used in other applications where detection of more than one analyte (i.e., nucleotide, protein, or fragments thereof) in a sample is desired.

The disclosed methods provide for all four nucleotide types to be simultaneously present in a sequence cycle, and to be distinguished by the use of minimal dyes and a single optical filter set.

In some embodiments, no more than three luminescent labels are used to determine the incorporation of all four nucleotide types that are present during a sequencing reaction, using two excitation wavelengths and a single emission detection filter.

In preferred embodiments, no more than two luminescent labels are used to determine the incorporation of all four nucleotide types that are all present during a sequencing reaction, using different excitation wavelengths and one detection filter.

It will be understood that reference to the use of a luminescent label includes the use of a plurality of different luminescent labels having the same or similar excitation and emission properties.

In preferred embodiments, the disclosed method is performed on a substrate (reaction surface), such as a glass, plastic, semiconductor chip or composite derived substrate, for example, within a flow cell.

Sequencing may be in a multiplex format, wherein multiple nucleic acid targets are detected and sequenced in parallel, for example in a flowcell or array type of format. The disclosed method is particularly advantageous when practicing parallel sequencing or massive parallel sequencing.

A combination of emitters may be selected for use in the disclosed methods based on the relative emission properties in response to radiation of different excitation wavelengths. For example, when irradiated with radiation of a specific excitation wavelength, the peak intensity of emission of a first emitter in the detection window (i.e. using a specific detection filter) may be significantly different to the peak intensity of emission of a second emitter, such that the emitters may be distinguished and/or identified based on their emission properties in response to radiation of a specific excitation wavelength.

Preferably, when irradiated with radiation of the specific excitation wavelength, the peak intensity of emission of the second emitter in the detection window is less than or equal to about 20%, preferably less than or equal to about 10%, such as less than or equal to about 5% or less than or equal to about 2% of the peak intensity of emission of the first emitter.

When irradiated with radiation of a second excitation wavelength, the peak intensity of emission of the first emitter in the detection window may be less than or equal to about 20%, preferably less than or equal to about 10%, such as less than or equal to about 5% or less than or equal to about 2% of the peak intensity of emission of the second emitter.

Emitters for use in the disclosed methods may be selected based on the relative properties of the emission in relation to the detection window used. Preferably, an emitter for use in any of the disclosed methods may have an emission peak which at 50% peak intensity overlaps with the detection window. Preferably, the emitter may have an emission peak which at 50% peak intensity overlaps with the detection window by at least 5 nm or 10 nm.

Emitters for use in any of the disclosed methods may have an emission peak which at 50% peak intensity encompasses at least 50%, at least 75%, or even the entire width of the detection window.

The skilled person will be aware of suitable resources, methods, and tools for determining the properties of the emission spectrum of an emitter, including specific properties, such as the proportion of the emission spectrum that resides between two specific wavelengths.

Emitters that are suitable for use in the disclosed methods may be selected on the basis that a significant proportion of the total area of the emission spectrum of the emitter resides within the detection window. For example, preferably at least 20% of the emission spectrum (i.e. 20% of the total area of the emission spectrum) of the emitter may be within the detection window. In some embodiments, at least 25%, 30%, or 35% of the emission spectrum of the emitter may be within the detection window.

A combination of different emitters may be selected for use in the disclosed methods based on a significant difference in the maximum absorption value (absorption peak). For example, the difference in absorption peaks of the first emitter and the second emitter may be at least 50 nm, at least 75 nm, or at least 100 nm.

The excitation wavelengths used in the disclosed methods may be selected based on the properties of the emitters used. Any suitable excitation wavelength may be used in the disclosed methods, the only consideration being the excitation properties of the emitters. For example, suitable excitation wavelengths may be selected from the following: 320 nm, 355 nm, 405 nm, 449 nm, 488 nm, 520 nm, 530 nm, 532 nm, 561 nm, 633 nm 635 nm, 640 nm, 650 nm, 660 nm, or 780 nm (in each case, each of these values may be varied by +/−2 nm, 5 nm, or 10 nm). A plurality of these excitation wavelengths may be used in combination depending on the excitation properties of the emitters used.

Emitters for use in any of the disclosed methods may be selected on the basis of relative brightness within the detection window or windows of the detection filter or filters used. The skilled person will be aware of various methods for determining the relative brightness of an emitter at a specific wavelength. As the skilled person will be aware, the emission properties of a given emitter depends on the efficiency with which it absorbs and emits photons, and its ability to undergo repeated excitation/emission cycles.

Absorption and emission efficiencies are most usefully quantified in terms of the molar extinction coefficient (EC) for absorption and the quantum yield (QY) for fluorescence. Both are constants under specific environmental conditions.

EC is a measure of how strongly a chemical species or substance absorbs light at a particular wavelength, and the value of EC is specified at a single wavelength, which is preferably the excitation wavelength of the emitter.

QY is a measure of the efficiency of photon emission through fluorescence, which is the loss of energy by a substance that has absorbed light via emission of a photon. QY may be defined as the ratio of the number of photons emitted to the number of photons absorbed, and is a measure of the total photon emission over the entire fluorescence spectral profile.

Fluorescence intensity per fluorophore (i.e. “brightness”) is proportional to the product of EC (preferably at the excitation wavelength) and QY.

A combination of different emitters may be selected for use in any of the disclosed methods on the basis of different relative brightness in the detection window (or at a specified wavelength) when irradiated with radiation of a specific excitation wavelength.

Preferably, when irradiated with radiation of the specific excitation wavelength, the brightness of a first emitter is at least 10× greater than the brightness of a second emitter in the detection window or at the specific wavelength. More preferably, when irradiated with radiation of the specific excitation wavelength, the brightness of the first emitter may be at least 20×, at least 50×, or at least 100× greater than the brightness of the second emitter in the detection window or at the specific wavelength.

In addition, it is preferable that when irradiated with radiation of a second excitation wavelength, the brightness of the second emitter is at least 10× greater than the brightness of the first emitter in the detection window or at the specific wavelength. Most preferably, when irradiated with radiation of the second excitation wavelength, the brightness of the second emitter is at least 20×, 50×, or 100× greater than the brightness of the first emitter.

### Sequencing Strategies

For purposes of illustration and not intended to limit embodiments, a general strategy sequencing cycle can be described by a sequence of steps. The following example is based on a sequence by synthesis sequencing reaction, however the disclosed methods are not limited to any particular sequencing reaction methodology.

The four nucleotide types A, C, T and G, typically modified nucleotides designed for sequencing reactions such as reversibly blocked (rb) nucleotides (e.g., rbA, rbT, rbC, rbG) wherein three of the four types of nucleotide are luminescently labelled, are simultaneously added, along with other reaction components, to the reaction surface (e.g., flowcell, chip, slide, etc.).

Following incorporation of a nucleotide into a growing sequence nucleic acid chain based on the target sequence, the reaction surface is irradiated with radiation of a first specific excitation wavelength and emission is recorded using a specific detection filter; this constitutes a first imaging event and a first luminescence detection pattern.

Following the first imaging event, the reaction surface is irradiated with radiation of a second specific excitation wavelength and emission is captured and recorded using the same detection filter, constituting a second imaging event (i.e., a second luminescence detection pattern).

The first and second excitation wavelengths are substantially different, and for example, may differ by at least 50 nm, 70 nm, or 100 nm. Specifically, the first and second excitation wavelengths are selected based on the excitation properties of the luminescent materials used in the sequencing method. Thus, the first excitation wavelength is selected in combination with first and second luminescent materials for use in the disclosed method such that when irradiated with radiation of the first excitation wavelength, the first luminescent material is significantly excited but the second luminescent material is not. Likewise, the second excitation wavelength is selected in combination with first and second luminescent materials such that when irradiated with radiation of the second excitation wavelength, the second luminescent material is significantly excited but the first luminescent material is not.

Various reagents present after the second imaging event are washed away in preparation for the next sequencing cycle. Exemplary chemical reagents that may be removed include, but are not limited to, blocking agents, luminescent labels, quenchers, cleavage reagents, or any other reagents that may directly or indirectly cause an identifiable and measurable change in luminescence, or which may inhibit the incorporation of the next correct nucleotide in the sequence.

The sequence of the target nucleic acid, for that particular cycle (i.e. the identity of the next cognate residue) is determined by identifying the nucleotide that has been incorporated from the luminescence patterns from the imaging events, as discussed below.

One approach for differentiating between the different strategies for detecting nucleotide incorporation in a sequencing reaction using the disclosed methods is by characterizing nucleotide incorporation in terms of the presence or relative absence, or levels in between, of luminescence transition that occurs during a sequencing cycle. As such, sequencing strategies can be exemplified by their luminescent profile for a sequencing cycle. Thus, “1” denotes a luminescent state in which a nucleotide is in a signal state (detectable by luminescence), “½” denotes a luminescent state in which a nucleotide is in a grey state, and “0” denotes a luminescent state in which a nucleotide is in a dark state (e.g. not detected or minimally detected at an imaging event). Luminescent states “1”, and in some embodiments “½”, may be considered to represent a positive signal, and luminescent state “0” may be considered to represent a negative signal or the absence of a positive signal.

Exemplary methods for detecting and determining nucleotide incorporation in a sequencing reaction are described below.

### Example Strategy Using Two Emitters

Two luminescent moieties, having different excitation properties, and one detection filter may be used to distinguish the incorporation of four different nucleotide types in a sequencing reaction.

Thus, the emission from two different emitters may be detected in the same detection filter and the emitters may be distinguished on the basis of having different excitation properties.

In some embodiments, the disclosed method comprises the use of a single detection filter and uses first and second luminescent labels having different excitation wavelengths and emission spectra which overlap with the detection window such that the emission from both labels are capable of being detected using the same specific detection filter.

In this example, four modified nucleotide triphosphates, in this case reversibly blocked nucleotide triphosphates (rbNTPs), are simultaneously added to a SBS reaction. The rbNTPs compete for incorporation into the growing nucleic acid strand during template directed extension of a primer. It is believed that competitive extension in the presence of a plurality of different nucleotides improves fidelity of incorporation as compared to adding nucleotides one at a time to a sequencing reaction. The four rbNTP types preferably possess a removable 3′-terminator. The terminator is removable, for example, by cleavage, thereby creating a nucleotide that is reversibly blocked and, once the terminator is removed, fully functional for further elongation (i.e., fully functional or ffNTPs).

Three of the four rbNTPs comprise luminescent labels. The first labeled rbNTP, which in this example is the thymine nucleotide (rbTTP), is labeled with a first luminescent label. The first luminescent label has a first excitation wavelength and has an emission that is capable of being detected using the specific detection filter.

A second labeled rbNTP, which in this example is the cytosine nucleotide or rbCTP, is labeled with a second luminescent label. The second luminescent label has a second excitation wavelength, that is different to the first excitation wavelength, and has an emission that is capable of being detected using the same specific detection filter that is capable of detecting the emission of the first luminescent label.

The absorption spectra of the first and second luminescent labels are such that when irradiated with radiation of the first excitation wavelength, the first luminescent label is excited but the second luminescent label is not excited or not significantly excited. Likewise, when irradiated with radiation of the second excitation wavelength, the second luminescent label is excited but the first luminescent label is not excited or not significantly excited.

A third labeled rbNTP, which in this example is the adenine nucleotide or rbATP, is labeled with both the first and second luminescent labels. In practice, there are a number of ways in which this dual labelling may be implemented. For example, each nucleotide molecule may be attached to two luminescent moieties, i.e. both the first and second luminescent labels, using linkers or any other suitable approach. In another example, each nucleotide molecule may be attached to a luminescent moiety that has excitation and emission spectra resembling a combination of the spectra of the first and second luminescent labels, such that the luminescent moiety is capable of being excited when irradiated with radiation of either the first or second excitation wavelength and has an emission that is capable of being detected using the specific detection filter. In another example, the population of adenine nucleotides comprises a combination of a first subpopulation of adenine nucleotides, which are attached to the first luminescent label, and a second subpopulation of adenine nucleotides, which are attached to the second luminescent label, such that the population of adenine nucleotides as a whole is capable of being excited when irradiated with radiation of both the first excitation wavelength and the second excitation wavelength, and when irradiated with radiation of either wavelength has an emission that is capable of being detected using the specific detection filter.

The fourth rbNTP, in this case guanine or rbGTP, lacks a luminescent label, or has a luminescent label that is not excited or not significantly excited when irradiated with radiation of either the first or second excitation wavelength, or does not have an emission that is capable of being detected by the specific detection filter, e.g. it has an emission spectrum that does not overlap with the detection window of the detection filter. Thus, the guanine nucleotide is considered a “dark” rbNTP and does not emit, or has diminished or minimal emission detectable by the specific detection filter.

With reference to the above, by “not excited or not significantly excited” it is meant that the level of excitation of the label is such that the resulting level of emission may be used to distinguish the nucleotide as being in the dark state, and providing a negative signal.

An exemplary detection scheme for a sequencing cycle for real time analysis of sequence by synthesis nucleotide incorporation utilizing the disclosed strategy comprises two imaging events, and identification of all four nucleotides requires no more than two imaging events.

Preferably, the luminescently labelled rbNTPs, rbTTP, rbATP and rbCTP and unlabelled (or otherwise dark) rbGTP are added simultaneously at the beginning of a sequencing cycle.

Electromagnetic radiation of the first excitation wavelength is applied to the sequencing reaction surface and a first image (image 1) is recorded using the specific detection filter.

The first image is capable of recording a positive signal in terms of emission (i.e. a signal state, “1”) for rbATP and rbTTP incorporations, which are labelled with the first luminescent label, but no positive signal, in terms of no luminescence or minimal luminescence (i.e. a dark state, “0”), for rbCTP or rbGTP incorporation, which do not possess the first luminescent label.

Following the first imaging event, radiation of the second excitation wavelength is applied to the sequencing reaction surface and a second image (image 2) is recorded using the same specific detection filter that was used to record the first image.

The second image is capable of recording a positive signal in terms of luminescence (i.e. a signal state, “1”) for rbATP and rbCTP incorporations, which are labelled with the second luminescent label, but no positive signal, in terms of no luminescence or minimal luminescence (i.e. a dark state, “0”), for rbTTP or rbGTP incorporation, which do not possess the second luminescent label.

Following the second image, a comparison of the luminescence detected in the first and second images (referred to as “luminescence transmissions”) may be used to determine the identity of the incorporated nucleotide in the sequence by synthesis reaction.

Thus, in this example, a signal state (1) in both the first and second imaging events identifies the incorporation of adenine in the sequence. A signal state (1) in the first imaging event transitioning to the dark state (0) in the second imaging event identifies the incorporation of thymine. A dark state (0) in the first imaging event transitioning to the signal state (1) in the second imaging event identifies the incorporation of cytosine. Finally, a dark state (0) in both the first and second imaging events identifies the incorporation of guanine in the sequence. This is demonstrated below in Table 1, in which “1” represents a signal state (positive signal) and “0” represents a dark state (lack of positive signal, or negative signal).

TABLE 1

| Image 1 | Image 2 | Identified nucleotide |
|---|---|---|
| 1 | 1 | A |
| 1 | 0 | T |
| 0 | 1 | C |
| 0 | 0 | G |

Each subsequent cycle follows the same pattern of:
1. Addition of reaction mixture and polymerase extension;
2. Irradiation with electromagnetic radiation of the first excitation wavelength, and image 1 recorded using the detection filter;
3. Irradiation with electromagnetic radiation of the second excitation wavelength and image 2 recorded using the same detection filter as image 1;
4. Removal or quenching of the luminescent labels and initiation of the next cycle.

Other steps can also be included per cycle including, but not limited to, deblocking, washing and/or additional steps used in sequence-by-synthesis methods known in the art.

Advantageously, the disclosed method uses the same detection filter throughout and there is no requirement for any modification of the nucleotides or any reagents (i.e., cleavage reagents, labeling reagents etc.) to be added between the imaging events.

In embodiments as discussed above in which the nucleotide that is labelled with both the first and second luminescent label (i.e. rbATP in the example above) comprises a combination of differentially labelled first and second subpopulations of the nucleotides, the level of luminescence for that nucleotide may be at 50% peak intensity relative to the nucleotides in the signal state (i.e. rbCTP and rbTTP in the example above). Thus, the nucleotide comprising differentially labelled subpopulations may be represented by a grey state (½) in both the first and second imaging events.

When applied to the example above, Table 2 shows the luminescence transitions that determine the identity of the incorporated nucleotide in the sequence by synthesis reaction (“1” represents a signal state, “½” represents a grey state, and “0” represents a dark state).

TABLE 2

| Image 1 | Image 2 | Identified nucleotide |
|---|---|---|
| ½ | ½ | A |
| 1 | 0 | T |
| 0 | 1 | C |
| 0 | 0 | G |

The disclosed methods are not limited to any particular luminescent labels and any two labels that may be excited when irradiated with radiation of different excitation wavelengths but having emission spectra which overlap with the detection window of the same filter may be used, in any combination of rbNTP-dye conjugate combination. Examples of dyes and derivatives thereof useful in embodiments described herein include, but are not limited to, those described below.

Thus, in some embodiments, when irradiated with radiation of the first excitation wavelength, emission from the first luminescent material is detectable at the detection filter, but there is no detectable emission, or substantially no detectable emission, from the second luminescent material. Likewise, in some embodiments, when irradiated with radiation of the second excitation wavelength, emission from the second luminescent material is detectable at the detection filter, but there is no detectable emission or substantially no detectable emission, from the first luminescent material.

The combination of first and second emitters may be selected for use in these embodiments such that, when irradiated with radiation of the first excitation wavelength, the peak intensity of emission of the second emitter in the detection window is less than or equal to about 20%, preferably less than or equal to about 10%, such as less than or equal to about 5% or less than or equal to about 2%, of the peak intensity of emission of the first emitter. When irradiated with radiation of the second excitation wavelength, the peak intensity of emission of the first emitter in the detection window is less than or equal to about 20%, preferably less than or equal to about 10%, such as less than or equal to about 5% or less than or equal to about 2%, of the peak intensity of emission of the second emitter.

For example, when irradiated with radiation of the first excitation wavelength, the peak intensity of emission of the second emitter in the detection window may be less than or equal to 20%, preferably less than or equal to about 10%, such as less than or equal to 5% or less than or equal to 2%, of the peak intensity of emission of the first emitter, and when irradiated with radiation of the second excitation wavelength, the peak intensity of emission of the first emitter in the detection window may be less than or equal to 20%, preferably less than or equal to about 10%, such as less than or equal to 5% or less than or equal to 2%, of the peak intensity of emission of the second emitter.

The first and/or second emitter for use in these embodiments may have an emission peak which at 50% peak intensity overlaps with the detection window. For example, the first and/or second emitter may have an emission peak which at 50% peak intensity overlaps with the detection window by at least 5 nm or 10 nm.

At least one of the first and/or second emitter may be selected on the basis of having an emission peak which at 50% peak intensity encompasses at least 50%, at least 75%, or even the entire width of the detection window.

The first and/or second emitter for use in these embodiments may be selected on the basis of having a significant proportion of the total area of its emission spectrum within the detection window. For example, at least one of the first and second emitters has an emission spectrum and at least 20% of the integrated intensity of the emission spectrum is within the detection window. For example, in some embodiments, at least 30%, 40%, 50%, 60%, 70%, 80%, or 90% of the integrated intensity of the emission spectrum is within the detection window.

The combination of first and second emitters may be selected for use in the disclosed methods on the basis of having a significantly different maximum absorption value (absorption peak). For example, the first emitter and the second emitter may have absorption peaks which differ by at least 50 nm, at least 75 nm, or at least 100 nm.

The combination of first and second emitters used in the disclosed method may be selected on the basis of the relative brightness of the different emitters within the detection window. Thus, a combination of emitters may be used in the disclosed method in which, at a specific detection wavelength within the detection window, when irradiated with radiation of the first excitation wavelength, the brightness of the first emitter is at least 10× greater than the brightness of the second emitter. Preferably, when irradiated with radiation of the first excitation wavelength, the brightness at the specific detection wavelength of the first emitter may be at least 20×, 50×, or 100× greater than is the brightness of the second emitter.

When irradiated with radiation of the second excitation wavelength, the brightness of the second emitter may be at least 10× greater than the brightness of the first emitter at the specific wavelength within the detection window. Preferably, when irradiated with radiation of the second excitation wavelength, the brightness at the specific detection wavelength of the second emitter is at least 20×, 50×, or 100× greater than the brightness of the first emitter.

The emission and absorption properties of different emitters are known in the art, and various suitable methods for determining the emission and absorption properties of different emitters will be known to the skilled person. Thus, the skilled person will be able to select appropriate combinations of emitters for use in the disclosed methods.

### Example Strategy Using Two Emitters

In some embodiments of the disclosed methods, four luminescent moieties, having different excitation properties, and two detection filters may be used to distinguish the incorporation of the four different nucleotide types in a sequencing reaction.

Thus, in these embodiments, the method comprises the use of two detection filters, having different detection windows, and uses first, second, third, and fourth luminescent labels, each label being used to identify a different nucleotide species.

In these examples, four modified nucleotide triphosphates, in this case reversibly blocked nucleotide triphosphates (rbNTPs), are each labelled with one of the first, second, third, and fourth luminescent labels, and simultaneously added to a SBS reaction.

The first labeled rbNTP, which in this example is the thymine nucleotide (rbTTP), is labeled with the first luminescent label. The first luminescent label has a first excitation wavelength and has an emission that is capable of being detected using the first detection filter, but not the second detection filter.

The second labeled rbNTP, which in this example is the cytosine nucleotide or rbCTP, is labeled with the second luminescent label. The second luminescent label has a second excitation wavelength and has an emission that, like the first luminescent label, is capable of being detected using the first detection filter, but not the second detection filter.

The third labeled rbNTP, which in this example is the adenine nucleotide or rbATP, is labeled with the third luminescent label. Like the first luminescent label, the third luminescent label is capable of being excited using radiation of the first excitation wavelength but not the second excitation wavelength. The third luminescent label has an emission that is capable of being detected using the second detection filter, but not the first detection filter.

The fourth labeled rbNTP, which in this example is the guanine nucleotide or rbGTP, is labeled with the fourth luminescent label. Like the second luminescent label, the fourth luminescent label is capable of being excited using radiation of the second excitation wavelength but not the first excitation wavelength. The fourth luminescent label has an emission that, like the third luminescent label, is capable of being detected using the second detection filter, but not the first detection filter.

Thus, the absorption spectra of the luminescent labels used in this method are such that when irradiated with radiation of the first excitation wavelength, the first and third luminescent labels are excited but the second and fourth luminescent labels are not excited or not significantly excited. When irradiated with radiation of the second excitation wavelength, the second and fourth luminescent labels are excited but the first and third luminescent labels are not excited or not significantly excited.

With reference to the above, by “not excited or not significantly excited” it is meant that the level of excitation of the label is such that the resulting level of emission may be used to distinguish the nucleotide as being in the dark state.

The emission spectra of the luminescent labels used in this method are such that the first and second luminescent labels have emission spectra that are capable of being detected using the first detection filter, but are not capable of being detected, or not significantly detected, using the second detection filter. The third and fourth luminescent labels have emission spectra that are capable of being detected using the second detection filter, but not capable of being detected, or not significantly detected, using the first detection filter.

With reference to the above, by “not detected or not significantly detected” it is meant that the level of emission of the label is such that the level of detected emission may be used to distinguish the nucleotide as being in the dark state.

An exemplary detection scheme for a sequencing cycle for real time analysis of sequence by synthesis nucleotide incorporation utilizing this method comprises four imaging events.

Preferably, the luminescently labelled rbNTPs, rbTTP, rbATP and rbCTP and rbGTP are added simultaneously at the beginning of a sequencing cycle.

Electromagnetic radiation of the first excitation wavelength is applied to the sequencing reaction surface and a first image (image 1) is recorded using the first detection filter.

The first image is capable of recording a positive signal in terms of emission (i.e. a signal state, “1”) for rbTTP incorporations, which is labelled with the first luminescent label, but no positive signal, in terms of no luminescence or minimal luminescence (i.e. a dark state, “0”), for rbCTP, rbATP, or rbGTP incorporation, which do not possess the first luminescent label.

Following the first imaging event, radiation of the second excitation wavelength is applied to the sequencing reaction surface and a second image (image 2) is recorded using the same specific first detection filter that was used to record the first image.

The second image is capable of recording a positive signal in terms of luminescence (i.e. a signal state, “1”) for rbCTP incorporations, which is labelled with the second luminescent label, but no positive signal, in terms of no luminescence or minimal luminescence (i.e. a dark state, “0”), for rbTTP, rbATP, or rbGTP incorporation, which do not possess the second luminescent label.

Following the second imaging event, radiation of the first excitation wavelength is applied to the sequencing reaction surface and a third image (image 3) is recorded using the second detection filter.

The third image is capable of recording a positive signal in terms of luminescence (i.e. a signal state, “1”) for rbATP incorporations, which is labelled with the third luminescent label, but no positive signal, in terms of no luminescence or minimal luminescence (i.e. a dark state, “0”), for rbTTP, rbCTP, or rbGTP incorporation, which do not possess the second luminescent label.

Finally, following the third imaging event, radiation of the second excitation wavelength is applied to the sequencing reaction surface and a fourth image (image 4) is recorded using the same specific second detection filter that was used to record the third image.

The fourth image is capable of recording a positive signal in terms of luminescence (i.e. a signal state, “1”) for rbGTP incorporations, which is labelled with the third luminescent label, but no positive signal, in terms of no luminescence or minimal luminescence (i.e. a dark state, “0”), for rbTTP, rbCTP, or rbATP incorporation, which do not possess the second luminescent label.

Following the fourth image, a comparison of the luminescence detected in the first, second, third, and fourth images (referred to as “luminescence transmissions”) may be used to determine the identity of the incorporated nucleotide in the sequence by synthesis reaction.

Thus, in this example, a signal state (1) in the first imaging event, and a signal state (0) in the second, third, and fourth imaging events, identifies the incorporation of thymine in the sequence.

A signal state (1) in the second imaging event, and a signal state (0) in the first, third, and fourth imaging events, identifies the incorporation of cytosine in the sequence.

A signal state (1) in the third imaging event, and a signal state (0) in the first, second, and fourth imaging events, identifies the incorporation of adenine in the sequence.

Finally, a signal state (1) in the fourth imaging event, and a signal state (0) in the first, second, and third, imaging events, identifies the incorporation of guanine in the sequence.

This is demonstrated below in Table 3, in which “1” represents a signal state (i.e. a positive signal) and “0” represents a dark state (i.e. the absence of a positive signal, or a negative signal).

TABLE 3

| | | | | Identified |
|---|---|---|---|---|
| Image 1 | Image 2 | Image 3 | Image 4 | nucleotide |
| 1 | 0 | 0 | 0 | T |
| 0 | 1 | 0 | 0 | C |
| 0 | 0 | 1 | 0 | A |
| 0 | 0 | 0 | 1 | G |

Other steps can also be included per cycle including, but not limited to, deblocking, washing and/or additional steps used in sequence-by-synthesis methods known in the art.

Advantageously, all of the disclosed methods comprise the detection of the emission from two different luminescent labels, that are excited when irradiated with radiation of different first and second excitation wavelengths, using a single detection filter to record an imaging event after irradiation with radiation of each of the first and second excitation wavelengths. The emitters are thus identified and distinguished on the basis of their absorption properties. There is no requirement for any modification of the nucleotides, or any reagents (i.e., cleavage reagents, labeling reagents etc.) to be added, between the imaging events.

Indeed, the method discussed above and illustrated in Table 3 involves performing this process twice. Thus, the same (i.e. the first) detection filter is used to detect the emission from two different (i.e. the first and second) luminescent labels excited using radiation of different (i.e. the first and second) excitation wavelengths. This process is then repeated, and the same (i.e. the second) detection filter is used to detect the emission from two different (i.e. the third and fourth) luminescent labels excited using radiation of different (i.e. the first and second) excitation wavelengths.

The disclosed methods are not limited to any particular luminescent labels and any combination of luminescent labels that may be excited using radiation of different first or second excitation wavelengths and having emission spectra which overlap with the detection window of the first or second detection filter may be used, in any combination of rbNTP-dye conjugate combination. Examples of dyes and derivatives thereof useful in embodiments described herein include, but are not limited to, those described below.

The combination of first, second, third, and fourth emitters may be selected for use in these embodiments such that, when irradiated with radiation of the first excitation wavelength, the peak intensity of emission of the second, third, and/or fourth emitters in the first detection window is less than or equal to about 20%, preferably less than or equal to about 10%, such as less than or equal to about 5% or less than or equal to about 2%, of the peak intensity of emission of the first emitter.

The combination of first, second, third, and fourth emitters may also be selected for use in these embodiments such that, when irradiated with radiation of the second excitation wavelength, the peak intensity of emission of the first, third, and/or fourth emitters in the first detection window is less than or equal to about 20%, preferably less than or equal to about 10%, such as less than or equal to about 5% or less than or equal to about 2%, of the peak intensity of emission of the second emitter.

The combination of first, second, third, and fourth emitters may also be selected for use in these embodiments such that, when irradiated with radiation of the first excitation wavelength, the peak intensity of emission of the first, second, and/or fourth emitters in the second detection window is less than or equal to about 20%, preferably less than or equal to about 10%, such as less than or equal to about 5% or less than or equal to about 2%, of the peak intensity of emission of the third emitter.

The combination of first, second, third, and fourth emitters may also be selected for use in these embodiments such that, when irradiated with radiation of the second excitation wavelength, the peak intensity of emission of the first, second, and/or third emitters in the second detection window is less than or equal to about 20%, preferably less than or equal to about 10%, such as less than or equal to about 5% or less than or equal to about 2%, of the peak intensity of emission of the fourth emitter.

For example, when irradiated with radiation of the first excitation wavelength, the peak intensity of emission of the second, third, and/or fourth emitters in the detection window of the first detection filter may be less than or equal to 20%, preferably less than or equal to about 10%, such as less than or equal to 5% or less than or equal to 2%, of the peak intensity of emission of the first emitter.

When irradiated with radiation of the second excitation wavelength, the peak intensity of emission of the first, third, and/or fourth emitters in the detection window of the first detection filter may be less than or equal to 20%, preferably less than or equal to about 10%, such as less than or equal to 5% or less than or equal to 2%, of the peak intensity of emission of the second emitter.

When irradiated with radiation of the first excitation wavelength, the peak intensity of emission of the first, second, and/or fourth emitters in the detection window of the second detection filter may be less than or equal to 20%, preferably less than or equal to about 10%, such as less than or equal to 5% or less than or equal to 2%, of the peak intensity of emission of the third emitter.

When irradiated with radiation of the second excitation wavelength, the peak intensity of emission of the first, second, and/or third emitters in the detection window of the second detection filter may be less than or equal to 20%, preferably less than or equal to about 10%, such as less than or equal to 5% or less than or equal to 2%, of the peak intensity of emission of the fourth emitter.

The first and/or second emitter for use in these embodiments may have an emission peak which at 50% peak intensity overlaps with the detection window of the first detection filter. For example, the first and/or second emitter may have an emission peak which at 50% peak intensity overlaps with the detection window of the first detection filter by at least 5 nm or 10 nm.

The third and/or fourth emitter for use in these embodiments may have an emission peak which at 50% peak intensity overlaps with the detection window of the second detection filter. For example, the third and/or fourth emitter may have an emission peak which at 50% peak intensity overlaps with the detection window of the second detection filter by at least 5 nm or 10 nm.

At least one of the first and/or second emitter may be selected on the basis of having an emission peak which at 50% peak intensity encompasses at least 50%, at least 75%, or even the entire width of the detection window of the first detection filter.

At least one of the third and/or fourth emitter may be selected on the basis of having an emission peak which at 50% peak intensity encompasses at least 50%, at least 75%, or even the entire width of the detection window of the second detection filter.

The first and/or second emitter for use in these embodiments may be selected on the basis of having a significant proportion of the total area of the emission spectrum within the detection window of the first detection filter, and similarly, the third and/or fourth emitter for use in these embodiments may be selected on the basis of having a significant proportion of the total area of the emission spectrum within the detection window of the second detection filter. For example, preferably at least 20% of the emission spectrum of the emitter (i.e. 20% of the total area of the emission spectrum) may be within the appropriate detection window. In some embodiments, at least 25%, 30%, or 35% of the emission spectrum of the emitter may be within the appropriate detection window.

The combinations of first and second, and/or third and fourth emitters may be selected for use in the disclosed methods on the basis of having a significantly different maximum absorption value (absorption peak). For example, the first emitter and the second emitter may have absorption peaks which differ by at least 50 nm, at least 75 nm, or at least 100 nm. The third emitter and the fourth emitter may have absorption peaks which differ by at least 50 nm, at least 75 nm, or at least 100 nm.

The combinations of first and second emitters used in the disclosed methods may be selected on the basis of the relative brightness of the emitters within the detection window of the first detection filter when irradiated with radiation of the first excitation wavelength. Thus, a combination of first and second emitters may be used in the disclosed methods in which, at a specific detection wavelength within the detection window of the first detection filter, when irradiated with radiation of the first excitation wavelength, the brightness of the first emitter is at least 10× greater than the brightness of the second emitter. Preferably, when irradiated with radiation of the first excitation wavelength, the brightness at the specific detection wavelength of the first emitter may be at least 20×, 50×, or 100× greater than the brightness of the second emitter.

The combinations of first and second emitters used in the disclosed methods may be selected on the basis of the relative brightness of the emitters within the detection window of the first detection filter when irradiated with radiation of the second excitation wavelength. Thus, a combination of first and second emitters may be used in the disclosed methods in which, at a specific detection wavelength within the detection window of the first detection filter, when irradiated with radiation of the second excitation wavelength, the brightness of the second emitter is at least 10× greater than the brightness of the first emitter. Preferably, when irradiated with radiation of the second excitation wavelength, the brightness at the specific detection wavelength of the second emitter may be at least 20×, 50×, or 100× greater than the brightness of the first emitter.

The combinations of third and fourth emitters used in the disclosed methods may be selected on the basis of the relative brightness of the emitters within the detection window of the second detection filter when irradiated with radiation of the first excitation wavelength. Thus, a combination of third and fourth emitters may be used in the disclosed methods in which, at a specific detection wavelength within the detection window of the second detection filter, when irradiated with radiation of the first excitation wavelength, the brightness of the third emitter is at least 10× greater than the brightness of the fourth emitter. Preferably, when irradiated with radiation of the first excitation wavelength, the brightness at the specific detection wavelength of the third emitter may be at least 20×, 50×, or 100× greater than the brightness of the fourth emitter.

The combinations of third and fourth emitters used in the disclosed methods may be selected on the basis of the relative brightness of the emitters within the detection window of the second detection filter when irradiated with radiation of the second excitation wavelength. Thus, a combination of third and fourth emitters may be used in the disclosed methods in which, at a specific detection wavelength within the detection window of the second detection filter, when irradiated with radiation of the second excitation wavelength, the brightness of the fourth emitter is at least 10× greater than the brightness of the third emitter. Preferably, when irradiated with radiation of the second excitation wavelength, the brightness at the specific detection wavelength of the fourth emitter may be at least 20×, 50×, or 100× greater than the brightness of the third emitter.

The emission and absorption properties of different emitters are known in the art, and various suitable methods for determining the emission and absorption properties of different emitters will be known to the skilled person. Thus, the skilled person will be able to select appropriate combinations of emitters for use in the disclosed methods.

### Luminescent Markers

The luminescent markers for use in any of the disclosed methods may be selected from any light-emitting materials known to the skilled person including non-polymeric and polymeric light-emitting materials.

In use, the luminescent marker may be dissolved or dispersed in the reaction mixture.

In some embodiments, the light-emitting marker is a particulate light-emitting marker comprising a light-emitting material.

The light-emitting material of the light-emitting marker may emit light having a peak wavelength in the range of 350-1000 nm.

A blue light-emitting material as described herein may have a photoluminescence spectrum with a peak of no more than 500 nm, preferably in the range of 400-500 nm, optionally 400-490 nm.

A green light-emitting material as described herein may have a photoluminescence spectrum with a peak of more than 500 nm up to 580 nm, optionally more than 500 nm up to 540 nm.

A red light-emitting material as described herein may have a photoluminescence spectrum with a peak of no more than more than 580 nm up to 950 nm, optionally up to 630 nm, optionally 585 nm up to 625 nm.

The light-emitting material may have a shift between excitation and emission maxima in the range of 20-400 nm.

UV/vis absorption spectra of light-emitting markers as described herein may be as measured in methanol solution or suspension using a Cary 5000 UV-vis-IR spectrometer.

Photoluminescence spectra of light-emitting particles as described herein may be measured in methanol solution or suspension using a Jobin Yvon Horiba Fluoromax-3.

Examples of non-polymeric light-emitting materials include, but are not limited to, fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, CoralHue mk02, DAPI, DiA, DiD, Dil, DiO, DiR, DRAQ5, DsRED, dTomato, DyeCycle dyes, EB, ECFP, EGFP, Emerald dyes, Eosin, EYFP, Fluo-dyes, Fura dyes, FVS dyes, Hoechst33258, Indo dyes, JC-1, Kusabira-Orange, Lucifer Yellow and derivatives such as Lucifer Yellow iodoacetamide, Lucifer Yellow CH, Magnesium Green, Marina Blue, mBanana, mCherry, mOrange, mPlum, mRaspberry, mStrawberry, mTangerine, methyl Coumarin, Mitotracker Red, Na-Green, Nile Red, Oregon Green, Pacific Blue, Pacific Orange, PE dyes, PerCP dyes, Picogreen, PI, QDot dyes, R718, Rho dyes, Rhodamine Red, Riboflavin, SNARF dyes, SYBR Green, SYTOX dyes, Texas Red, TO-Pro dyes, TOTO dyes, V450, V500, Via-probe dyes, YO-Pro dyes, YOYO dyes, ZsGreen, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malacite green, stilbene, DEG dyes, NR dyes, CF dyes, near-infrared dyes and others known in the art.

A light-emitting polymer as described herein may be a fluorescent or phosphorescent light-emitting polymer. In use, the light-emitting polymer may be dissolved in the reaction mixture or a particulate light-emitting marker comprising or consisting of the light-emitting polymer may be dispersed in the reaction mixture.

Examples of polymeric light-emitting materials that may be suitable for use in the disclosed methods include, but are not limited to, Horizon Brilliant dyes by Becton Dickinson, Super Bright Dyes by ThermoFisher, StarBright dyes by Bio-Rad, and KIRAVIA Dyes by Sony. Other suitable polymeric light emitting materials are discussed below.

The light-emitting polymer may be a homopolymer or may be a copolymer comprising two or more different repeat units.

The light-emitting polymer may comprise light-emitting groups in the polymer backbone, pendant from the polymer backbone or as end groups of the polymer backbone. In the case of a phosphorescent polymer, a phosphorescent metal complex, preferably a phosphorescent iridium complex, may be provided in the polymer backbone, pendant from the polymer backbone or as an end group of the polymer backbone.

The light-emitting polymer may have a non-conjugated backbone or may be a conjugated polymer. By “conjugated polymer” is meant a polymer comprising repeat units in the polymer backbone that are directly conjugated to adjacent repeat units. Conjugated light-emitting polymers include, without limitation, polymers comprising one or more of arylene, heteroarylene and vinylene groups conjugated to one another along the polymer backbone.

The light-emitting polymer may comprise host repeat units and emissive repeat units wherein the emissive repeat units and host repeat units. In use, the host repeat units may absorb excitation energy and transfer it to the emissive repeat units.

In some embodiments, first and second light-emitting polymers may be provided in which the first and second light-emitting polymers have the same or similar emissive repeat units and different host repeat units, or a different composition of host repeat units, such that emission from the first and second polymers is similar but absorption peaks are at different wavelengths. In some embodiments, the first polymer contains a first host repeat unit and an emissive repeat unit and the second polymer contains the first host repeat unit, the emissive repeat unit and an intermediate repeat unit having a band gap between that of the first host repeat unit and the emissive repeat unit.

The light-emitting polymer may have a linear, branched or crosslinked backbone.

The light-emitting polymer may comprise one or more repeat units in the backbone of the polymer substituted with one or more substituents selected from non-polar and polar substituents.

Preferably, the light-emitting polymer comprises at least one polar substituent. The one or more polar substituents may be the only substituents of said repeat units, or said repeat units may be further substituted with one or more non-polar substituents, optionally one or more C₁₋₄₀ hydrocarbyl groups. The repeat unit or repeat units substituted with one or more polar substituents may be the only repeat units of the polymer or the polymer may comprise one or more further co-repeat units wherein the or each co-repeat unit is unsubstituted or is substituted with non-polar substituents, optionally one or more C₁₋₄₀ hydrocarbyl substituents.

C₁₋₄₀ hydrocarbyl substituents as described herein include, without limitation, C₁₋₂₀ alkyl, unsubstituted phenyl and phenyl substituted with one or more C₁₋₂₀ alkyl groups.

As used herein a “polar substituent” may refer to a substituent, alone or in combination with one or more further polar substituents, which renders the light-emitting polymer with a solubility of at least 0.01 mg/ml in an alcoholic solvent, optionally in the range of 0.01-10 mg/ml. Optionally, solubility is at least 0.1 or 1 mg/ml. The solubility is measured at 25° C. Preferably, the alcoholic solvent is a C₁₋₁₀ alcohol, more preferably methanol.

Polar substituents are preferably substituents capable of forming hydrogen bonds or ionic groups.

In some embodiments, the light-emitting polymer comprises polar substituents of formula —O(R³O)ₜ—R⁴ wherein R³ in each occurrence is a C₁₋₁₀ alkylene group, optionally a C₁₋₅ alkylene group, wherein one or more non-adjacent, non-terminal C atoms of the alkylene group may be replaced with O, R⁴ is H or C₁₋₅ alkyl, and t is at least 1, optionally 1-10. Preferably, t is at least 2. More preferably, t is 2 to 5. The value of t may be the same in all the polar groups of formula —O(R³O)ₜ—R⁴. The value of t may differ between polar groups of the same polymer.

By “C₁₋₅ alkylene group” as used herein with respect to R³ is meant a group of formula —(CH₂)_{f}— wherein f is from 1-5.

Preferably, the light-emitting polymer comprises polar substituents of formula —O(CH₂CH₂O)ₜ—R⁴ wherein t is at least 1, optionally 1-10 and R⁴ is a C₁₋₅ alkyl group, preferably methyl. Preferably, t is at least 2. More preferably, t is 2 to 5, most preferably t is 3.

In some embodiments, the light-emitting polymer comprises polar substituents of formula —N(R⁵)₂, wherein R⁵ is H or C₁₋₁₂ hydrocarbyl. Preferably, each R⁵ is a C₁₋₁₂ hydrocarbyl.

In some embodiments, the light-emitting polymer comprises polar substituents which are ionic groups which may be anionic, cationic or zwitterionic. Preferably the ionic group is an anionic group.

Exemplary anionic groups are —COO⁻, a sulfonate group; hydroxide; sulfate; phosphate; phosphinate; or phosphonate.

An exemplary cationic group is —N(R⁵)₃⁺ wherein R⁵ in each occurrence is H or C₁₋₁₂ hydrocarbyl. Preferably, each R⁵ is a C₁₋₁₂ hydrocarbyl.

A light-emitting polymer comprising cationic or anionic groups comprises counterions to balance the charge of these ionic groups.

An anionic or cationic group and counterion may have the same valency, with a counterion balancing the charge of each anionic or cationic group.

The anionic or cationic group may be monovalent or polyvalent. Preferably, the anionic and cationic groups are monovalent.

The light-emitting polymer may comprise a plurality of anionic or cationic polar substituents wherein the charge of two or more anionic or cationic groups is balanced by a single counterion. Optionally, the polar substituents comprise anionic or cationic groups comprising di- or trivalent counterions.

The counterion is optionally a cation, optionally a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

The counterion is optionally an anion, optionally a halide; a sulfonate group, optionally mesylate or tosylate; hydroxide; carboxylate; sulfate; phosphate; phosphinate; phosphonate; or borate.

In some embodiments, the light-emitting polymer comprises polar substituents selected from groups of formula —O(R³O)ₜ—R⁴, groups of formula —N(R⁵)₂, groups of formula OR⁴ and/or ionic groups. Preferably, the light-emitting polymer comprises polar substituents selected from groups of formula —O(CH₂CH₂O)ₜR⁴, groups of formula —N(R⁵)₂, and/or anionic groups of formula —COO⁻. Preferably, the polar substituents are selected from the group consisting of groups of formula —O(R³O)ₜ—R⁴, groups of formula —N(R⁵)₂, and/or ionic groups. Preferably, the polar substituents are selected from the group consisting of polyethylene glycol (PEG) groups of formula —O(CH₂CH₂O)ₜ⁴, groups of formula —N(R⁵)₂, and/or anionic groups of formula —COO⁻. R³, R⁴, R⁵, and t are as described above.

Optionally, the backbone of the light-emitting polymer is a conjugated polymer. Optionally, the backbone of the conjugated light-emitting polymer comprises repeat units of formula (III):

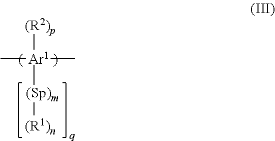

wherein Ar¹ is an arylene group or heteroarylene group; Sp is a spacer group; m is 0 or 1; R¹ independently in each occurrence is a polar substituent; n is 1 if m is 0 and n is at least 1, optionally 1, 2, 3 or 4, if m is 1; R² independently in each occurrence is a non-polar substituent; p is 0 or a positive integer, optionally 1, 2, 3 or 4; q is 0 or a positive integer, optionally 1, 2, 3 or 4; and wherein Sp, R¹ and R² may independently in each occurrence be the same or different.

Preferably, m is 1 and n is 2-4, more preferably 4. Preferably p is 0.

Ar¹ of formula (III) is optionally a C₆₋₂₀ arylene group or a 5-20 membered heteroarylene group. Ar¹ is preferably a C₆₋₂₀ arylene group, optionally phenylene, fluorene, benzofluorene, phenanthrene, naphthalene or anthracene, more preferably fluorene or phenylene, most preferably fluorene.

Sp-(R¹)n may be a branched group, optionally a dendritic group, substituted with polar groups, optionally —NH₂ or —OH groups, for example polyethyleneimine.

Preferably, Sp is selected from:
C₁₋₂₀ alkylene or phenylene-C₁₋₂₀ alkylene wherein one or more non-adjacent C atoms may be replace with O, S, N or C═O;
a C₆₋₂₀ arylene or 5-20 membered heteroarylene, more preferably phenylene, which, in addition to the one or more substituents R¹, may be unsubstituted or substituted with one or more non-polar substituents, optionally one or more C₁₋₂₀ alkyl groups.

“alkylene” as used herein means a branched or linear divalent alkyl chain.

“non-terminal C atom” of an alkyl group as used herein means a C atom other than the methyl group at the end of an n-alkyl group or the methyl groups at the ends of a branched alkyl chain.

More preferably, Sp is selected from:
C₁₋₂₀ alkylene wherein one or more non-adjacent C atoms may be replaced with O, S or CO; and
a C₆₋₂₀ arylene or a 5-20 membered heteroarylene, even more preferably phenylene, which may be unsubstituted or substituted with one or more non- polar substituents.

R¹ may be a polar substituent as described anywhere herein. Preferably, R¹ is:
a polyethylene glycol (PEG) group of formula —O(CH₂CH₂O)ₜR⁴ wherein t is at least 1, optionally 1-10 and R⁴ is a C₁₋₅ alkyl group, preferably methyl;
a group of formula —N(R⁵)₂, wherein R⁵ is H or C₁₋₁₂ hydrocarbyl; or
an anionic group of formula —COO⁻.

In the case where n is at least two, each R¹ may independently in each occurrence be the same or different. Preferably, each R¹ attached to a given Sp group is different.

In the case where p is a positive integer, optionally 1, 2, 3 or 4, the group R² may be selected from:
alkyl, optionally C₁₋₂₀ alkyl; and
aryl and heteroaryl groups that may be unsubstituted or substituted with one or more substituents, preferably phenyl substituted with one or more C₁₋₂₀ alkyl groups;
a linear or branched chain of aryl or heteroaryl groups, each of which groups may independently be substituted, for example a group of formula —(Ar³)ₛ wherein each Ar³ is independently an aryl or heteroaryl group and s is at least 2, preferably a branched or linear chain of phenyl groups each of which may be unsubstituted or substituted with one or more C₁₋₂₀ alkyl groups; and
a crosslinkable-group, for example a group comprising a double bond such and a vinyl or acrylate group, or a benzocyclobutane group.

Preferably, each R², where present, is independently selected from C₁₋₄₀ hydrocarbyl, and is more preferably selected from C₁₋₂₀ alkyl; unusubstituted phenyl; phenyl substituted with one or more C₁₋₂₀ alkyl groups; and a linear or branched chain of phenyl groups, wherein each phenyl may be unsubstituted or substituted with one or more substituents.

A polymer as described herein may comprise or consist of only one form of the repeating unit of formula (III) or may comprise or consist of two or more different repeat units of formula (III).

Optionally, the polymer comprising one or more repeat units of formula (III) is a copolymer comprising one or more co-repeat units.

If co-repeat units are present then the repeat units of formula (III) may form between 0.1-99 mol % of the repeat units of the polymer, optionally 50-99 mol % or 80-99 mol %. Preferably, the repeat units of formula (I) form at least 50 mol % of the repeat units of the polymer, more preferably at least 60, 70, 80, 90, 95, 98 or 99 mol %. Most preferably the repeat units of the polymer consist of one or more repeat units of formula (I).

The or each repeat unit of the polymer may be selected to produce a desired colour of emission of the polymer.

Arylene repeat units of the polymer include, without limitation, fluorene, preferably a 2,7-linked fluorene; phenylene, preferably a 1,4-linked phenylene; naphthalene, anthracene, indenofluorene, phenanthrene and dihydrophenanthrene repeat units.

The polystyrene-equivalent number-average molecular weight (Mn) measured by gel permeation chromatography of the light-emitting polymers described herein may be in the range of about 1×10³ to 1×10⁸, and preferably 1×10⁴ to 5×10⁶. The polystyrene-equivalent weight-average molecular weight (Mw) of the polymers described herein may be 1×10³ to 1×10⁸, and preferably 1×10⁴ to 1×10⁷.

Polymers as described herein are suitably amorphous polymers.

A particulate light-emitting marker as described herein may be, without limitation, a micro- or nano-particulate light-emitting marker.

In some embodiments, the particulate light emitting marker comprises or consists of a quantum dot. Exemplary light-emitting quantum dot materials include, without limitation, metal chalcogenides. Quantum dots include, without limitation, core, core-shell and alloyed quantum dots.

In some embodiments, the particulate light-emitting marker is a collapsed light-emitting polymer.

In some embodiments, the light-emitting particles of the particulate light-emitting marker comprise a light-emitting material and a matrix. The light-emitting material may be a fluorescent or phosphorescent light-emitting material. The light-emitting material be selected from polymeric or non-polymeric light-emitting materials as described anywhere herein.

Preferably, the light-emitting particles as described herein have a number average diameter of no more than 500 nm or 400 nm in methanol as measured by dynamic light scattering (DLS) using a Malvern Zetasizer Nano ZS (Details of measurement in the Examples). Preferably the particles have a number average diameter of between 5-500 nm, optionally 10-200 nm, preferably between 20-150 nm, as measured by a Malvern Zetasizer Nano ZS. The inventors have found that light emitting particles having an average diameter of less than 50 nm, such as 20-40 nm are preferred. In addition, the inventors have produced light emitting particles having a diameter of 30 nm which have an extinction coefficient that is at least two orders of magnitude higher than that of a typical small molecule dye. Particles of this size are also ideally suited for use in current sequencing methods, for example, using substrate beads having diameters in the range of 1-2 μm, or nanowells in the range of 200-600 nm.

The matrix of a light-emitting particle comprising a matrix and a light-emitting material may at least partially isolate the light-emitting material from the surrounding environment. This may limit any effect that the external environment may have on the lifetime of the light-emitting material.

In some embodiments, the particle comprises the light-emitting material homogenously distributed through the matrix.

In some embodiments, the particle may have a particulate core and, optionally, a shell wherein at least one of the core and shell contains the light-emitting material.

In the case where the light-emitting material is a polymer, polymer chains of the light-emitting polymer may extend across some or all of the thickness of the core and/or shell. Polymer chains may be contained within the core and/or shell or may protrude through the surface of the core and/or shell.

In some embodiments, the particle comprises a core comprising or consisting of the light-emitting polymer and a shell comprising or consisting of the matrix.

In some embodiments, the particle core consists of the matrix and the light-emitting material. In some embodiments, the particle core comprises at least one further material, for example a host material configured to absorb excitation energy from an energy source, e.g. a light source, and transfer energy to the transferring energy to the light-emitting material.

Exemplary polymeric matrix materials include, without limitation, polystyrene and homopolymers or copolymers of (alkyl)acrylic acids. A polymeric matrix material may be crosslinked, e.g. a crosslinked chitosan-polyacrylic acid polymer. The polymer matrix may be a self-assembled micelle or vesicle comprising lipid or polymer surfactants. The polymer matrix is preferably an inorganic oxide, optionally silica, alumina or titanium dioxide. The polymer matrix is more preferably silica.

In some embodiments the light-emitting material may be covalently bound, directly or indirectly, to the matrix material. In some embodiments, the light-emitting material may be mixed with (i.e. not covalently bound to) a matrix material. Preferably, the io matrix is not covalently bound to the light-emitting material, in which case there is no need for the matrix material and/or the light-emitting material to be substituted with reactive groups for forming such covalent bonds, e.g. during formation of the particles.

In some embodiments, a silica matrix as described herein may be formed by polymerisation of a silica monomer in the presence of the light-emitting material, for example as described in WO 2018/060722, the contents of which are incorporated herein by reference.

In some embodiments, the polymerisation comprises bringing a solution of silica monomer into contact with an acid or a base. The acid or base may be in solution. The light-emitting material may be in solution with the acid or base and/or the silica monomer before the solutions are mixed. Optionally, the solvents of the solutions are selected from water, one or more C₁₋₈ alcohols or a combination thereof.

Polymerising a matrix monomer in the presence of a light-emitting polymer may result in one or more chains of the polymer encapsulated within the particle and/or one or more chains of the polymer extending through a particle.

The particles may be formed in a one-step polymerisation process.

Optionally, the silica monomer is an alkoxysilane, preferably a trialkoxy or tetra-alkoxysilane, optionally a C₁₋₁₂ trialkoxy or tetra-alkoxysilane, for example tetraethyl orthosilicate. The silica monomer may be substituted only with alkoxy groups or may be substituted with one or more groups.

Optionally, at least 0.1 wt % of total weight of the particle core consists of the light-emitting material. Preferably at least 1, 10, 25 wt % of the total weight of the particle core consists of the light-emitting material.

Optionally at least 50 w t% of the total weight of the particle core consists of the matrix. Preferably at least 60, 70, 80, 90, 95, 98, 99, 99.5, 99.9 wt % of the total weight of the particle core consists of the matrix.

### EXAMPLES

### Hypothetical Example 1

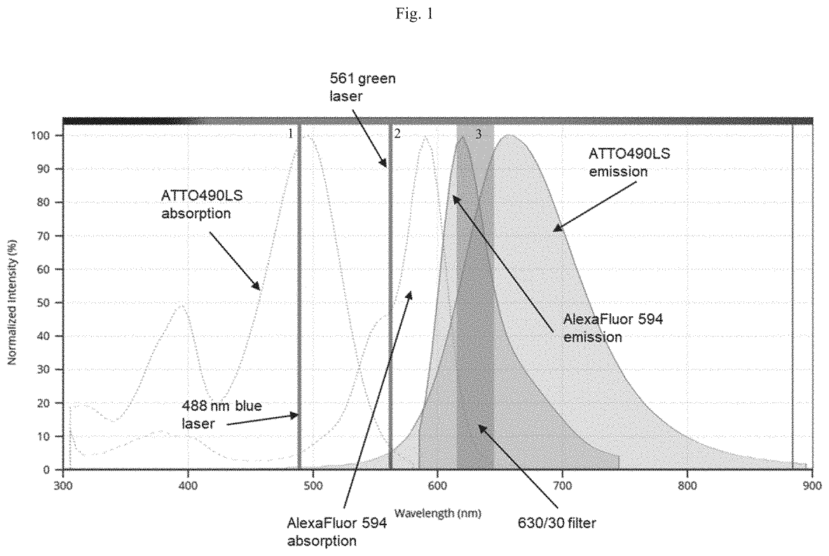

A combination of ATTO 490LS and Alexa Fluor 594 may be used as the first and second emitters respectively. The absorption and emission spectra of these fluorophores is shown in FIG. 1.

As the skilled person will appreciate from FIG. 1, the use of a first excitation wavelength of 488 nm, which is shown in FIG. 1 using a dark vertical solid line labelled with reference numeral 1, will excite the ATTO 490LS label but not the Alexa Fluor 594 label.

The luminescence emission from the excitation of the ATTO 490LS label may be detected using an orange 630/30 detection filter, which is shown in FIG. 1 using a solid shaded vertical bar labelled with reference numeral 3.

The use of a second excitation wavelength of 561 nm, which is shown in FIG. 1 using a pale vertical solid line labelled with reference numeral 2, will excite the Alexa Fluor 594 label but not the ATTO 490LS label.

The luminescence emission from the excitation of the Alexa Fluor 594 label may also be detected using the same orange 630/30 detection filter.

In a hypothetical example of a sequencing by synthesis in accordance with the present disclosure, rbNTPs are labelled as indicated in Table 4.

TABLE 4

| Nucleotide | Label |
|---|---|
| rbTTP | ATTO 490LS |
| rbCTP | Alexa Fluor 594 |
| rbATP | First subpopulation: ATTO490LS |
| | Second subpopulation: Alexa Fluor 594 |
| rbGTP | No label |

A reaction mixture, comprising all four of the rbNTPs, is used in an SBS method in the usual way.

After incorporation and polymerization of the next correct nucleotide, the reaction surface is washed and laser light of 488 nm wavelength (the first excitation wavelength) is applied to the sequencing reaction surface. A first image (image 1) is recorded using an orange 630/30 detection filter.

Following the first imaging event, laser light of 561 nm wavelength (the second excitation wavelength) is applied to the sequencing reaction surface. A second image (image 2) is recorded using the same orange 630/30 detection filter that was used to record the first image.

Following the second image, a comparison of the luminescence detected in the first and second images may be used to determine the identity of the incorporated nucleotide in the sequence by synthesis reaction in accordance with the following table 5.

TABLE 5

| Image 1 | Image 2 | Identified nucleotide |
|---|---|---|
| 1 (or 1/2) | 1 (or 1/2) | A |
| 1 | 0 | T |
| 0 | 1 | C |
| 0 | 0 | G |

Thus, the identity of all four different nucleotides is established using two different excitation wavelengths and a single detection filter.

Following the removal or quenching of the fluorescent labels, a further sequencing cycle is initiated, using nucleotides labelled using the same emitters as indicated in Table 4.

### Hypothetical Examples 2-4

In Hypothetical Examples 2-4, the method set out in Hypothetical Example 1 is repeated using further combinations of first and second emitters, including in particular, all combinations of small molecule and polymeric emitters.

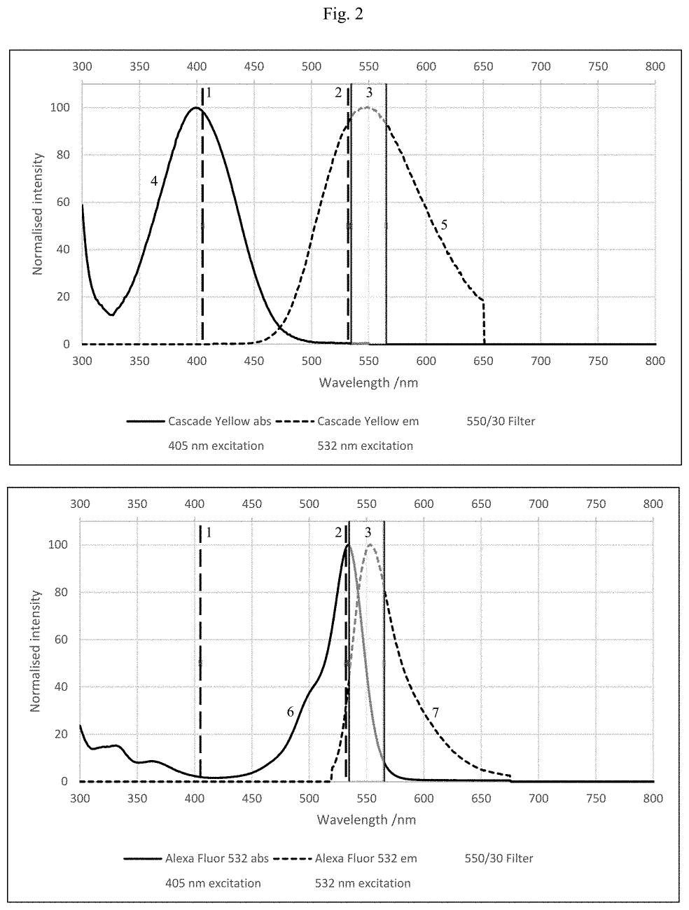

The excitation and emission spectra of the first and second emitters used in each of Examples 2-4 are shown in FIGS. 2-4 respectively, on separate graphs for ease of reference. Each graph pair has the same excitation sources and filter window to show that the dyes can be individually excited but their emission overlaps with the same detection filter. In each Figure, dashed vertical lines are used to represent the first excitation wavelength 1 and the second excitation wavelength 2, and a shaded zone bordered by solid vertical lines represents the filter window 3.

All of the graphs shown in FIGS. 2-4 show the absorption spectrum of the indicated emitter using a solid line, and the emission spectrum of the indicated emitter using a dotted line.

### Hypothetical Example 2

Hypothetical Example 2 relates to the use of a combination of two small molecule emitters, Cascade Yellow and Alexa Fluor 532, as the first and second labels respectively.

The upper panel of FIG. 2 shows the absorption spectrum 4 and emission spectrum 5 of the first emitter, Cascade Yellow.

The lower panel of FIG. 2 shows the absorption spectrum 6 and emission spectrum 7 of the second emitter, Alexa Fluor 532.

As the skilled person will appreciate from FIG. 2, the use of a first excitation wavelength (1) of 405 nm will excite the Cascade Yellow label 4, but not the Alexa Fluor 532 label 6.

The use of a second excitation wavelength (2) of 532 nm will excite the Alexa Fluor 532 label 6, but not the Cascade Yellow label 4.

The luminescence emission from the excitation of both labels may be detected using a 530/30 detection filter 3.

Table 6 shows the percentage of the peak intensity of emission of each emitter in response to the first and second excitation wavelengths.

TABLE 6

| | % max at | % max at |
|---|---|---|
| Emitter | 405 nm excitation | 532 nm excitation |
| Cascade Yellow | 98.4 | 0.3 |
| Alexa Fluor 532 | 0.5 | 93.3 |

It is clear from Table 6 that when irradiated with radiation of 405 nm (the first excitation wavelength), the emission intensity of Alexa Fluor 532 label (the second emitter) in the detection window is 0.5% of the peak intensity.

When irradiated with radiation of 532 nm (the second excitation wavelength), the emission intensity of the Cascade Yellow label (the first emitter) in the detection window is 0.3% of the peak intensity.

### Hypothetical Example 3

Hypothetical Example 3 relates to the use of a combination of a polymeric emitter, Brilliant Violet 510 (BV510), and a small molecule emitter, Alexa Fluor 488, as the first and second labels respectively.

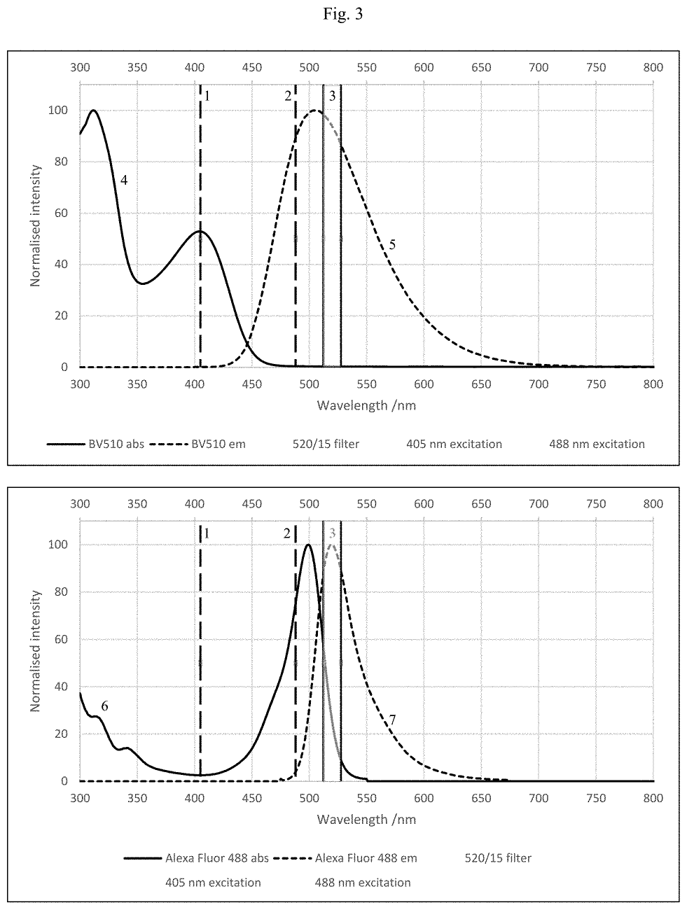

The upper panel of FIG. 3 shows the absorption spectrum 4 and emission spectrum 5 of the first emitter, BV510.

The lower panel of FIG. 3 shows the absorption spectrum 6 and emission spectrum 7 of the second emitter, Alexa Fluor 488.

The use of a first excitation wavelength (1) of 405 nm will excite the Brilliant Violet 510 label 4, but not the Alexa Fluor 488 label 6.

The use of a second excitation wavelength (2) of 488 nm will excite the Alexa Fluor 488 label 6, but not the Brilliant Violet 510 label 4.

The luminescence emission from the excitation of both labels may be detected using a 520/15 detection filter 3.

### Hypothetical Example 4

Hypothetical Example 4 relates to the use of a combination of two polymeric emitters, Brilliant Violet 510 (BV510) and Brilliant Blue 515 (BB515), as the first and second labels respectively.

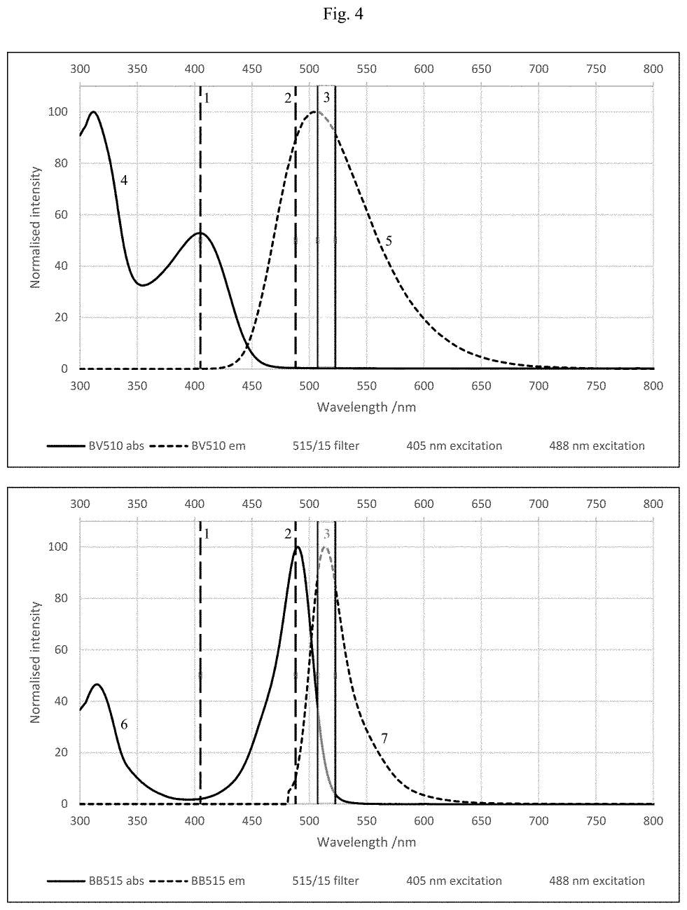

is The upper panel of FIG. 4 shows the absorption spectrum 4 and emission spectrum 5 of the first emitter, BV510.

The lower panel of FIG. 4 shows the absorption spectrum 6 and emission spectrum 7 of the second emitter, BB515.

The use of a first excitation wavelength (1) of 405 nm will excite the BV510 label 4, but not the BB515 label 6.

The use of a second excitation wavelength (2) of 488 nm will excite the BB515 label 6, but not the BV510 label 4.

The luminescence emission from the excitation of both labels may be detected using a 515/15 detection filter 3.

### Hypothetical Example 5

In Hypothetical Example 5, the method set out in Hypothetical Example 1 is extended to further include the use of third and fourth emitters and a second detection filter.

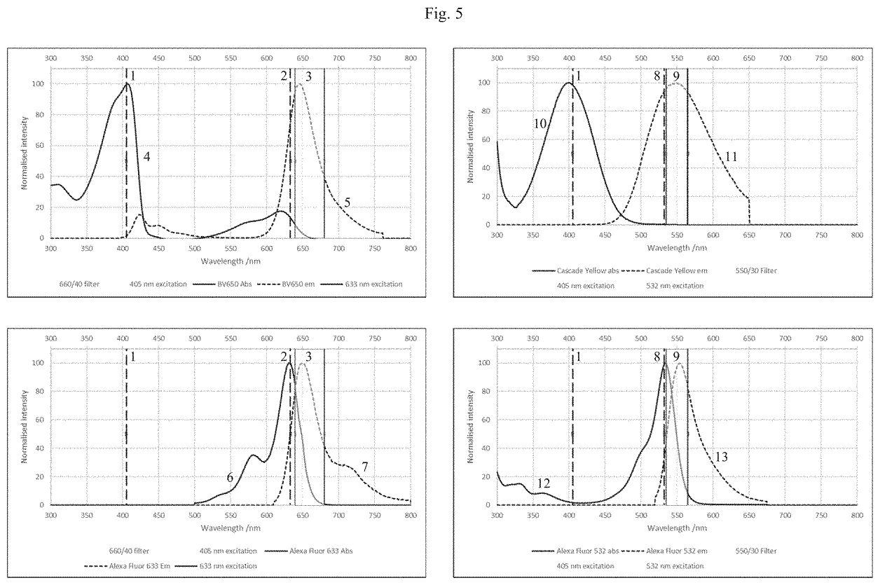

The upper and lower left-hand panels of FIG. 5 shows the excitation and emission spectra of the first and second emitters, illustrating that the first and second emitters can be individually excited, using radiation of different excitation wavelengths, but their emission can be detected using the same first detection filter.

Specifically, the upper left-hand panel of FIG. 5 shows the absorption spectrum 4 and emission spectrum 5 of the first emitter, BV650.

The lower left-hand panel of FIG. 5 shows the absorption spectrum 6 and emission spectrum 7 of the second emitter, Alexa Fluor 633.

Irradiation with radiation of a first excitation wavelength (1) of 405 nm will excite the BV650 label 4, but not the Alexa Fluor 633 label 6.

Irradiation with radiation of a second excitation wavelength (2) of 633 nm will excite the Alexa Fluor 633 label 6, but not the BV650 label 4.

The luminescence emission from the excitation of both of the first and second emitters may be detected using a 660/40 detection filter 3 (a first detection filter).

The upper and lower right-hand panels of FIG. 5 shows the excitation and emission spectra of the third and fourth emitters, illustrating that the third and fourth emitters can be individually excited, using radiation of different excitation wavelengths, but their emission can be detected using the same second detection filter.

Specifically, the upper right-hand panel of FIG. 5 shows the absorption spectrum 10 and emission spectrum 11 of the third emitter, Cascade Yellow.

The lower right-hand panel of FIG. 5 shows the absorption spectrum 12 and emission spectrum 13 of the fourth emitter, Alexa Fluor 532.

Irradiation with radiation of the first excitation wavelength (1) of 405 nm will excite the Cascade Yellow label 10, but not the Alexa Fluor 532 label 12.

Irradiation with radiation of a third excitation wavelength (8) of 532 nm will excite the Alexa Fluor 532 label 12, but not the Cascade Yellow label 10.

The luminescence emission from the excitation of both labels may be detected using a 550/30 detection filter 9.

In a hypothetical example of a sequencing by synthesis in accordance with the present disclosure, rbNTPs are labelled as indicated in Table 7.

TABLE 7

| Nucleotide | Label |
|---|---|
| rbTTP | First emitter - BV650 |
| rbCTP | Second emitter - Alexa Fluor 633 |
| rbATP | Third emitter - Cascade Yellow |
| rbGTP | Fourth emitter - Alexa Fluor 532 |

A reaction mixture, comprising all four of the rbNTPs, is used in an SBS method in the usual way.

After incorporation and polymerization of the next correct nucleotide, the reaction surface is washed and laser light of 405 nm wavelength (the first excitation wavelength) is applied to the sequencing reaction surface. A first image (image 1) is recorded using a 660/40 detection filter (the first detection filter).

Following the first imaging event, laser light of 633 nm wavelength (the second excitation wavelength) is applied to the sequencing reaction surface. A second image (image 2) is recorded using the same 660/40 first detection filter that was used to record the first image.

Following the second imaging event, laser light of 405 nm wavelength (the first excitation wavelength) is applied to the sequencing reaction surface. A third image (image 3) is recorded using a 550/30 detection filter (the second detection filter).

Following the third imaging event, laser light of 532 nm wavelength (the third excitation wavelength) is applied to the sequencing reaction surface. A fourth image (image 4) is recorded using the same 550/30 second detection filter that was used to record the third image.

Following the fourth image, a comparison of the luminescence detected in the first, second, third, and fourth images may be used to determine the identity of the incorporated nucleotide in the sequence by synthesis reaction in accordance with the following table 8.

TABLE 8

| | | | | Identified |
|---|---|---|---|---|
| Image 1 | Image 2 | Image 3 | Image 4 | nucleotide |
| 1 | 0 | 0 | 0 | T |
| 0 | 1 | 0 | 0 | C |
| 0 | 0 | 1 | 0 | A |
| 0 | 0 | 0 | 1 | G |

Thus, the identity of all four different nucleotides is established using three different excitation wavelengths and two different detection filters.

Following the removal or quenching of the fluorescent labels, a further sequencing cycle is initiated, using nucleotides labelled using the same emitters as indicated in Table 7.

The skilled person will appreciate that the disclosed methods are not limited to these specifically disclosed combinations of emitters, and based on the guidance provided above will be able to select various alternative suitable combinations of emitters for use in the disclosed methods.

1. A method for determining the sequence of a polynucleotide comprising detecting in a sequencing reaction the incorporation of a first, second, third, or fourth nucleotide using radiation of first and second excitation wavelengths and a single photodetector having a detection window comprising a range of wavelengths,
wherein when excited:
the first nucleotide comprises a first emitter but not a second emitter;
the second nucleotide comprises the second emitter but not the first emitter;
the third nucleotide comprises the first and second emitters; and
the fourth nucleotide comprises neither the first nor second emitter
when irradiated with radiation of the first excitation wavelength, the peak intensity of emission of the second emitter in the detection window is ≤20% of the peak intensity of emission of the first emitter, and
when irradiated with radiation of the second excitation wavelength, the peak intensity of emission of the first emitter in the detection window is ≤20% of the peak intensity of emission of the second emitter.

2. A method as claimed in claim 1, wherein:
(i) in response to radiation of the first excitation wavelength, the brightness of the first emitter in the detection window is at least 10× greater than the brightness of the second emitter in the detection window; and/or
(ii) in response to radiation of the second excitation wavelength, the brightness of the second emitter in the detection window is at least 10× greater than the brightness of the first emitter in the detection window.

3. A method as claimed in claim 1, wherein the first and second excitation wavelengths differ by at least 50 nm.

4. A method as claimed in claim 1, wherein at least one of the first and second emitters has an emission peak which at 50% peak intensity:
(i) overlaps by at least 5 nm with the detection window; and/or
(ii) encompasses the width of the detection window.

5. A method as claimed in claim 1, wherein at least one of the first and second emitters has an emission spectrum:
(i) which overlaps with the detection window; and/or
(ii) wherein at least 20% of the integrated intensity of the emission spectrum is within the detection window.

6. A method as claimed in claim 1, wherein at least one of the first and second emitters has an emission peak, measured in terms of FWHM, which is entirely within the detection window.

7. A method as claimed in claim 1, wherein the difference in absorption peaks of the first emitter and the second emitter is at least 50 nm.

8. A method as claimed in claim 1, wherein the method further comprises:
a first imaging event after irradiation with radiation of the first excitation wavelength providing a first luminescent detection pattern at the photodetector; and
a second imaging event after irradiation with radiation of the second excitation wavelength providing a second luminescent detection pattern at the photodetector,
wherein the combination of first and second luminescent detection patterns is used identify the nucleotide that is incorporated into the polynucleotide.

9. A method as claimed in claim 8, wherein
a positive signal in the first but not the second luminescence detection pattern is indicative of the incorporation of the first nucleotide into the polynucleotide;
a positive signal in the second but not the first luminescence detection pattern is indicative of the incorporation of the second nucleotide into the polynucleotide;
a positive signal in both the first and second luminescence detection patterns is indicative of the incorporation of the third nucleotide into the polynucleotide; and
a positive signal in neither the first nor second luminescence detection pattern is indicative of the incorporation of the fourth nucleotide into the polynucleotide.

10. A method as claimed in claim 1, wherein the photodetector comprises a band pass filter which has a width of ≤60 nm.

11. A method as claimed in claim 1, wherein the method comprises a plurality of imaging events, and wherein the nucleotides and associated emitters are identical and are not chemically or physically modified, added, removed, replaced, or masked between imaging events.

12. A method as claimed in claim 1, wherein either of the first and/or second emitters comprises a particulate emitter.

13. A method as claimed in claim 1, wherein either of the first and/or second emitters is dissolved in the sample.

14. A composition for use in a method of sequencing a nucleic acid, the composition comprising:
a first species of nucleotide comprising a first emitter;
a second species of nucleotide comprising a second emitter;
a third species of nucleotide comprising both the first and second emitters; and
a fourth species of nucleotide comprising neither the first nor second emitter,
wherein the first emitter is excitable when irradiated with radiation of a first excitation wavelength,
wherein the second emitter is excitable when irradiated with radiation of a second excitation wavelength,
wherein luminescence emission from the first emitter when irradiated with radiation of the first excitation wavelength and from the second emitter when irradiated with radiation of the second excitation wavelength is detectable at a single detection wavelength,
wherein when irradiated with radiation of the first excitation wavelength, the peak intensity of emission of the second emitter at the detection wavelength is ≤20% of the peak intensity of emission of the first emitter, and
wherein when irradiated with radiation of the second excitation wavelength, the peak intensity of emission of the first emitter at the detection wavelength is ≤20% of the peak intensity of emission of the second emitter.
